(19) United States
(12) Patent Application Publication
(10) Pub. No.: US 2003/0129203 A1
(43) Pub. Date: Jul. 10, 2003

(54) Mutant recombinant adeno-associated viruses

(75) Inventors: Manuel Vega, Vigneux-sur-Seine (FR); Lila Drittanti, Vigneux-sur-Seine (FR); Marjorie Flaux, Ris-Orangis (FR)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: Nautilus Biotech S.A.

(21) Appl. No.: 10/022,390

(22) Filed: Dec. 17, 2001

Related U.S. Application Data
(60) Provisional application No. 60/315,382, filed on Aug. 27, 2001.

(51) Int. Cl. A61K 31/70; A01N 43/04; C12P 21/06; C12P 19/34; A61K 48/00; A01N 63/00; A61K 39/12; A61K 39/23; A61K 39/235; C12N 7/00; C12N 7/01; C12N 15/00; C12N 15/09; C12N 15/63; C12N 15/70; C12N 15/74

(52) U.S. Cl. 424/233.1; 514/44; 424/199.1; 424/205.1; 424/93.2; 435/69.1; 435/91.33; 435/91.1; 435/235.1; 435/320.1

(57) ABSTRACT

Processes and systems for the high throughput directed evolution of peptides and proteins, particularly those that act in complex biological settings, are provided. The proteins and peptides include, but are not limited to, intracellular proteins, messenger/signaling/hormone proteins and viral proteins. Also provided is a rational method for generating protein variants and also a method for titering viruses.

### RELATED APPLICATIONS

Benefit of priority under 35 U.S.C. §119(e) is claimed to U.S. provisional application Serial No. 60/315,382, filed Aug. 27, 2001, to Manuel Vega and Lila Drittanti, entitled “HIGH THROUGHPUT DIRECTED EVOLUTION BY RATIONAL MUTAGENESIS.” The subject matter of the provisional application is incorporated in its entirety by reference thereto.

### FIELD OF INVENTION

Mutant adeno-associated viruse Rep proteins, recombinant viruses that express the proteins and nucleic acid molecule encoding the Rep proteins are provided. Uses of the recombinant viruses for treatment of diseases and a vectors for gene therapy are also provided.

### BACKGROUND

Adeno-associated virus (AAV) is a defective and non-pathogenic parvovirus that requires co-infection with either adenovirus or a herpes virus, which provide helper functions, for its growth and multiplication. There is an extensive body of knowledge regarding AAV biology and genetics (see, e.g., Weitzman et al. (1996) J. Virol. 70: 2240-2248 (1996); Walker et al. (1997) J. Virol. 71:2722-2730; Urabe et al. (1999) J. Virol. 23:2682-2693; Davis et al. (2000) J. Virol. 23:74:2936-2942; Yoon et al. (2001) J. Virol. 75:3230-3239; Deng et al. (1992) Anal Biochem 200:81-85; Drittanti et al. (2000) Gene Therapy 7:924-929; Srivastava et al (1983) J. Virol. 45:555-564; Hermonat et al. (1984) J. Virol. 51:329-339; Chejanovsky et al. (1989) Virology 173:120-128; Chejanovsky et al. (1990) J. Virol. 64:1764-1770; Owens et al. (1991) Virology 184:14-22; Owens et al. (1992) J. Virol. 66:1236-1240; Qicheng Yang et al. (1992) J. Virol. 66:6058-6069; Qicheng Yang et al. (1993) J. Virol. 67:4442-4447; Owens et al. (1993) J. Virol. 62:997-1005; Sirkka et al. (1994) J. Virol. 68:2947-2957; Ramesh et al. (1995) Biochem. Biophy. Res. Com. Vol 210 (3), 717-725; Sirkka (1995) J. Virol. 69:6787-6796; Sirkka et al. (1996) Biochem. Biophy. Res. Com. 220:294-299; Ryan et al (1996) J. Virol. 70:1542-1553; Weitzman et al. (1996) J. Virol. 70:2440-2448; Walker et al. (1997) J. Virol. 71:2722-2730; Walker et al. (1997) J. Virol. 71:6996-7004; Davis et al. (1999) J. Virol. 73:2084-2093; Urabe et al. (1999) J. Virol. 73:2682-2693; Gavin et al. (1999) J. Virol. 73:9433-9445; Davis et al. (2000) J. Virol. 74:2936-2942; Pei Wu et al. (2000) J. Virol. 74:8635-8647; Alessandro Marcello et al. (2000) J. Virol. 74:9090-9098). AAV are members of the family Parvoviridae and are assigned to the genus Dependovirus. Members of this genus are small, non-enveloped, icosahedral with linear and single-stranded DNA genomes, and have been isolated from many species ranging from insects to humans.

AAV can either remain latent after integration into host chromatin or replicate following infection. Without co-infection, AAV can enter host cells and preferentially integrate at a specific site on the q arm of chromosome 19 in the human genome.

The AAV genome contains 4975 nucleotides and the coding sequence is flanked by two inverted terminal repeats (ITRs) on either side that are the only sequences in cis required for viral assembly and replication. The ITRs contain palindromic sequences, which form a hairpin secondary structure, containing the viral origins of replication. The ITRs are organized in three segments: the Rep binding site (RBS), the terminal resolution site (TRS), and a spacer region separating the RBS from the TRS.

Regulation of AAV genes is complex and involves positive and negative regulation of viral transcription. For example, the regulatory proteins Rep 78 and Rep 68 interact with viral promoters to establish a feedback loop (Beaton et al. (1989) J. Virol. 63:4450-4454; Hermonat (1994) Cancer Lett 81:129-136). Expression from the p5 and p19 promoters is negatively regulated in trans by these proteins. Rep 78 and 68, which are required for this regulation, have bind to inverted terminal repeats (ITRs; Ashktorab et al. (1989) J. Virol. 63:3034-3039) in a site- and stand-specific manner, in vivo and in vitro. This binding to ITRs induces a cleavage at the TRS and permits the replication of the hairpin structure, thus, illustrating the Rep helicase and endonuclease activities (Im et al. (1990) Cell 61:447-457; and Walker et al. (1997) J. Virol. 71:6996-7004), and the role of these non-structural proteins in the initial steps of DNA replication (Hermonat et al. (1984) J. Virol. 52:329-339). Rep 52 and 40, the two minor forms of the Rep proteins, do not bind to ITRs and are dispensable for viral DNA replication and site-specific integration (im et al. (1992) J. Virol. 66:1119-112834; Ni et al. (1994) J. Virol. 68:1128-1138.

The genome (see, FIG. 1) is organized into two open reading frames (ORFs, designated left and right) that encode structural capsid proteins (Cap) and non-structural proteins (Rep). There are three promoters: p5 (from nucleotides 255 to 261: TATTTAA), p19 (from nucleotide 843 to 849: TATTTAA) and p40 (from nucleotides 1822 to 1827: ATATAA). The right-side ORF (see FIG. 1) encodes three capsid structural proteins (Vp 1-3). These three proteins, which are encoded by overlapping DNA, result from differential splicing and the use of an unusual initiator codon (Cassinoti et al. (1988) Virology 167:176-184). Expression of the capsid genes is regulated by the p40 promoter. Capsid proteins VP1, VP2 and VP3 intiate from the p40 promoter. VP1 uses an alternate splice acceptor at nucleotide 2201; whereas VP2 and VP3 are derived from the same transcription unit, but VP2 use an ACG triplet as an initiation codon upstream from the start of VP3. On the left side of the genome, two promoters p5 and p19 direct expression of four regulatory proteins. The left flanking sequence also uses a differential splicing mechanism (Mendelson et al. (1986) J. Virol 60:823-832) to encode the Rep proteins, designated Rep 78, 68, 52 and 40 on the basis molecular weight. Rep 78 and 68 are translated from a transcript produced from the p5 promoter and are produced from the unspliced and spliced form, respectively, of the transcript. Rep 52 and 40 are the translation products of unspliced and spliced transcripts from the p19 promoter.

AAV and rAAV have many applications, including use as a gene transfer vector, for introducing heterologous nucleic acid into cells and for genetic therapy. Advances in the production of high-titer rAAV stocks to the transition to human clinical trials have been made, but improvement of rAAV production will be complemented with special attention to clinical applications of rAAV vectors as successful gene therapy approach. Productivity of rAAV (i.e. the amount of vector particles that can be obtained per unitary manufacturing operation) is one of the rate limiting steps in the further development of rAAV as gene therapy vector. Methods for high throughput production and screening of rAAV have been developed (see, e.g., Drittanti et al. (2000) Gene Therapy 7:924-929) Briefly, as with the other steps in methods provided herein, the plasmid preparation, transfection, virus productivity and titer and biological activity assessment are intended to be performed in automatable high throughput format, such as in a 96 well or loci formats (or other number of wells or multiples of 96, such as 384, 1536 . . . 9600, 9984 . . . well or loci formats).

### SUMMARY

Mutant AAV Rep proteins, nucleic acid molecules encoding such proteins, and rAAV that encode the proteins are provided. Among the rep proteins are those that result in increased rAAV production in rAAV that encode such mutants, thereby, among a variety of advantages, offer a solution to the need in the gene therapy industry to increase the production therapeutic vectors without up-scaling manufacturing. Methods of gene therapy using the rAAV are provided.

Directed evolution methods provided in co-pending U.S. provisional application Serial No. 60/315,382, filed as U.S. application Serial No. ______ (attorney dkt no. 37851-911), and described herein have been used to identify amino acid “hit” positions in adeno-associated virus (AAV) rep proteins that are relevant for AAV or rAAV production. Those amino acid positions are selected such that a change in the amino acid leads to a change in protein activity either to lower activity or to higher activity compared to native-sequence Rep proteins. The hit positions were then used to generate further mutants designated “leads.” Provided herein are the resulting mutant rep proteins that result in either higher or lower levels of AAV or rAAV virus compared to the wild-type (native) Rep protein(s). Nucleic acid molecules that encode the mutant Rep proteins are also provided

Also provided are rAAV that contain the nucleic acid molecules and methods that use the rAAV to produce the mutant Rep. Cell-free (in vitro) and intracellular methods are provided. Cells containing the rAAV are also provided.

Among the Rep mutants provided herein, in addition to Rep mutants that enhance AAV production, are those that inhibit papillomavirus (PV) and PV-associated diseases, including certain cancers and human immunodeficiency virus (HIV) and HIV-associated diseases. Methods of treating such diseases are provided.

### DESCRIPTION OF THE FIGURES

FIG. 1 shows the genetic map of AAV, including the location of promoters, and transcripts; amino acid 1 of the Rep 78 gene is at nucleotie 321 in the AAV-2 genome.

FIGS. 2A and 2B depict “HITS” and “LEADS” respectively for identification of AAV rep mutants “evolved” for increased activity.

FIGS. 3A and 3B show the alignment of amino acid sequences of Rep78 among AAV-1; AAV-6; AAV-3; AAV-3B; AAV-4; AAV-2; AAV-5 sequences, respectively; the hit positions with 100 percent homology among the serotypes are bolded italics, where the position is different (compared to AAV-2, no. 6 in the Figure) in a particular serotype, it is in bold; a sequence indicating relative conservation of sequences among the serotypes is labeled “C”.

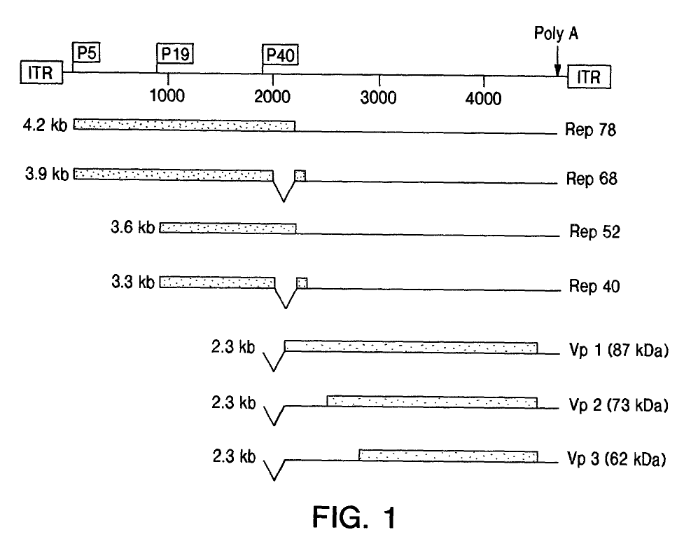

### LEGEND

1 is AAV-1; 2 is AAV-6, 3 is AAV-3, 4 is AAV-3B,

5 is AAV-4, 6 is AAV-2, and 7 is AAV-5;

“.” where the amino acid is present ≧20%;

“:” where the amino acid is present ≧40%;

“+” where the amino acid is present ≧60%;

“*” where the amino acid is present ≧80%; and

where the amino acid is the same amongst all serotypes depicted it is represented by its single letter code.

### DETAILED DESCRIPTION

A. Definitions

Unless defined otherwise, all technical and scientific terms used herein have the same meaning as is commonly understood by one of skill in the art to which this invention belongs. All patents, patent applications, published applications and publications, Genbank sequences, websites and other published materials referred to throughout the entire disclosure herein are, unless noted otherwise, incorporated by reference in their entirety. In the event that there are a plurality of definitions for terms herein, those in this section prevail.

As used herein, directed evolution refers to mehods that adapt” natural proteins or protein domains to work in new chemical or biological environments and/or to elicit new functions. It is more a more broad-based technology than DNA shuffling.

As used herein, high-throughput screening (HTS) refers to processes that test a large number of samples, such as samples of test proteins or cells containing nucleic acids encoding the proteins of interest to identify structures of interest or the identify test compounds that interact with the variant proteins or cells containing them. HTS operations are amenable to automation and are typically computerized to handle sample preparation, assay procedures and the subsequent processing of large volumes of data.

As used herein, DNA shuffling is a PCR-based technology that produces random rearrangements between two or more sequence-related genes to generate related, although different, variants of given gene.

As used herein, “hits” are mutant proteins that have an alteration in any attribute, chemical, physical or biological property in which such alteration is sought. In the methods herein, hits are generally generated by systematically replacing each amino acid in a the protein or a domain thereof with a selected amino acid, typically Alanine, Glycine, Serine or any amino acid, as long as each residue is replaced with the same residue. Hits may be generated by other methods known to those of skill in the art tested by the highthroughput methods herein. For purposes herein a Hit typically has activity with respect to the function of interest that differs by at least 10%, 20%, 30% or more from the wild type or native protein. The desired alteration, which is generally a reduction in activity, will depend upon the function or property of interest.

As used herein, “leads” are “hits” whose activity has been optimized for the particular attribute, chemical, physical or biological property. In the methods herein, leads are generally produced by systematically replacing the hit loci with all remaining 18 amino acids, and identifying those among the resulting proteins that have a desired activity. The leads may be further optimized by replacement of a plurality of “hit” residues. Leads may be generated by other methods known to those of skill in the and tested by the highthroughput methods herein. For purposes herein a lead typically has activity with respect to the function of interest that differs from the native activity, by a desired amount and is at by at least 10%, 20%, 30% or more from the wild type or native protein. Generally a Lead will have an activity that is 2 to 10 or more times the native protein for the activity of interest. As with hits, the change in the activity is dependent upon the activity that is “evolved.”The desired alteration will depend upon the function or property of interest.

As used herein, MOI is multiplicity of infection.

As used herein, ip, with reference to a virus or recombinant vector, refers to a titer of infectious particles.

As used herein, pp refers to the total number of vector (or virus) physical particles

As used herein, biological and pharmacological activity includes any activity of a biological pharmaceutical agent and includes, but is not limited to, biological efficiency, transduction efficiency, gene/transgene expression, differential gene expression and induction activity, titer, progeny productivity, toxicity, citotoxicity, immunogenicity, cell proliferation and/or differentiation activity, anti-viral activity, morphogenetic activity, teratogenetic activity, pathogenetic activity, therapeutic activity, tumor supressor activity, ontogenetic activity, oncogenetic activity, enzymatic activity, pharmacological activity, cell/tissue tropism and delivery.

As used herein, “output signal” refers to parameters that can be followed over time and, if desired, quantified. For example, when a virus infects or is introduced into a cell, the cell containing the virus undergoes a number of changes. Any such change that can be monitored and used to assess infection, is an output signal, and the cell is referred to as a reporter cell; the encoding nucleic acid is referred to as a reporter gene, and the construct that includes the encoding nucleic acid is a reporter construct. Output signals include, but are not limited to, enzyme activity, fluorescence, luminescence, amount of product produced and other such signals. Output signals include expression of a viral gene or viral gene product, including heterologous genes (transgenes) inserted into the virus. Such expression is a function of time (“t”) after infection, which in turn is related to the amount of virus used to infect the cell, and, hence, the concentration of virus (“s”) in the infecting composition. For higher concentrations the output signal is higher. For any particular concentration, the output signal increases as a function of time until a plateau is reached. Output signals may also measure the interaction between cells, expressing heterologous genes, and biological agents

As used herein, adeno-associated virus (AAV) is a defective and non-pathogenic parvovirus that requires co-infection with either adenovirus or herpes virus for its growth and multiplication, able of providing helper functions. A variety of serotypes are known, and contemplated herein. Such serotypes include, but are not limited to: AAV-1 (Genbank accession no. NC002077; accession no. VR-645); AAV-2 (Genbank accession no. NC001401; accession no. VR-680); AAV-3 (Genbank accession no. NC001729; acession no. VR-681); AAV-3b (Genbank accession no. NC001863); AAV-4 (Genbank accession no. NCO01 829; ATCC accession no. VR-646); AAV-6 (Genbank accession no.NCO01 729); and avian associated adeno-virus (ATCC accession no. VR-1449). The preparation and use of AAVs as vectors for gene expression in vitro and for in vivo use for gene therapy is well known (see, e.g., U.S. Pat. Nos. 4,797,368, 5,139,941, 5,798,390 and 6,127,175; Tessier et al. (2001) J. Virol. 75:375-383; Salvetti et al. (1998) Hum Gene Ther 20:695-706; Chadeuf et al. (2000) J Gene Med 2:260-268).

As used herein, the activity of a Rep protein or of a capsid protein refers to any biological activity that can be assessed. In particular, herein, the activity assessed for the rep proteins is the amount (i.e., titer) of AAV produced by a cell.

As used herein, the Hill equation is a mathematical model that relates the concentration of a drug (i.e., test compound or substance) to the response being measured

$$y = \frac{{{y}_{max} [ D ]}^{x}}{{[ D ]}^{n} + {[ {D}_{50} ]}^{n}} ,$$

where y is the variable being measured, such as a response, signal, yₘₐₓ is the maximal response achievable, [D] is the molar concentration of a drug, [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs. log [D] (M). The slope is n, where a slope of greater than 1 indicates cooperativity among binding sites, and a slope of less than 1 can indicate heterogeneity of binding. This general equation has been employed for assessing interactions in complex biological systems (see, published International PCT application No. WO 01/44809 based on PCT n° PCT/FR00/03503, see, also, EXAMPLES).

As used herein, in the Hill-based analysis (published International PCT application No. WO 01/44809 based on PCT n° PCT/FR00/03503), the parameters, π,K,T,ε,η,θ, are as follows:

π potency of the biological agent acting on the assay (cell-based) system;

K constant of resistance of the assay system to elicit a response to a biological agent;

ε is global efficiency of the process or reaction triggered by the biological agent on the assay system;

T is the apparent titer of the biological agent;

θ is the absolute titer of the biological agent; and

η is the heterogeneity of the biological process or reaction.

In particular, as used herein, the parameters π (potency) or K (constant of resistance) are used to respectively assess the potency of a test agent to produce a response in an assay system and the resistance of the assay system to respond to the agent.

As used herein, ε(efficiency), is the slope at the inflexion point of the Hill curve (or, in general, of any other sigmoidal or linear approximation), to asses the efficiency of the global reaction (the biological agent and the assay system taken together) to elicit the biological or pharmacological response.

As used herein, T (apparent titer) is used to measure the limiting dilution or the apparent titer of the biological agent.

As used herein, θ (absolute titer), is used to measure the absolute limiting dilution or titer of the biological agent.

As used herein, η (heterogeneity) measures the existence of discontinuous phases along the global reaction, which is reflected by an abrupt change in the value of the Hill coefficient or in the constant of resistance.

As used herein, a library of mutants refers to a collection of plasmids or other vehicles that carrying (encoding) the gene variants, such that individual plasmid or other vehicles carry individual gene variants. When a library of proteins is contemplated, it will be so-stated.

As used herein, a “reporter cell” is the cell that “reports”, i.e., undergoes the change, in response to introduction of the nucleic acid infection and, therefore, it is named here a reporter cell.

As used herein, “reporter” or “reporter moiety” refers to any moiety that allows for the detection of a molecule of interest, such as a protein expressed by a cell. Reporter moieties include, but are not limited to, for example, fluorescent proteins, such as red, blue and green fluorescent proteins; lacZ and other detectable proteins and gene products. For expression in cells, nucleic acid encoding the reporter moiety can be expressed as a fusion protein with a protein of interest or under to the control of a promoter of interest.

As used herein, a titering virus increases or decreases the output signal from a reporter virus, which is a virus that can be detected, such as by a detectable label or signal.

As used herein, phenotype refers to the physical, physiological or other manifestation of a genotype (a sequence of a gene). In methods herein, phenotypes that result from alteration of a genotype are assessed.

As used herein, activity refers to the function or property to be evolved An active site refers to a site(s) responsible or that participates in conferring the activity or function. The activity or active site evolved (the function or property and the site conferring or participating in conferring the activity) may have nothing to do with natural activities of a protein. For example, it could be an ‘active site’ for conferring immunogenicity (immunogenic sites or epitopes) on a protein.

As used herein, the amino acids, which occur in the various amino acid sequences appearing herein, are identified according to their known, three-letter or one-letter abbreviations (see, Table 1). The nucleotides, which occur in the various nucleic acid fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are presumed to be in the “L” isomeric form. Residues in the “D” isomeric form, which are so-designated, can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in the following Table:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions are preferably made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, nucleic acids include DNA, RNA and analogs thereof, including protein nucleic acids (PNA) and mixture thereof. Nucleic acids can be single or double stranded. When referring to probes or primers, optionally labeled, with a detectable label, such as a fluorescent or radiolabel, single-stranded molecules are contemplated. Such molecules are typically of a length such that they are statistically unique of low copy number (typically less than 5, preferably less than 3) for probing or priming a library. Generally a probe or primer contains at least 14, 16 or 30 contiguous of sequence complementary to or identical a gene of interest. Probes and primers can be 10, 14, 16, 20, 30, 50, 100 or more nucleic acid bases long.

As used herein, by homologous means about greater than 25% nucleic acid sequence identity, preferably 25% 40%, 60%, 80%, 90% or 95%. The intended percentage will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

As used herein, a nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are, for example, at least 80%, 85%, 90%, 95%, 96%, 97%, 98% or 99%, “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)), and CLUSTALW. For sequences displaying a relatively high degree of homology, alignment can be effected manually by simpling lining up the sequences by eye and matching the conserved portions.

Therefore, as used herein, the term “identity” represents a comparison between a test and a reference polypeptide or polynucleotide. For example, a test polypeptide may be defined as any polypeptide that is 90% or more identical to a reference polypeptide.

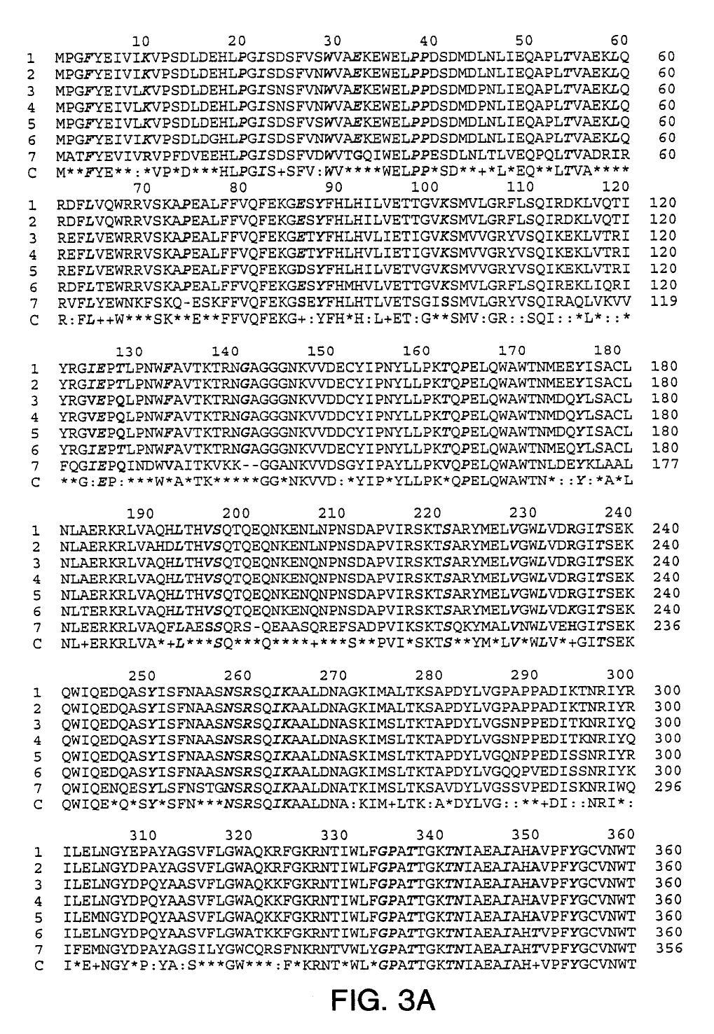

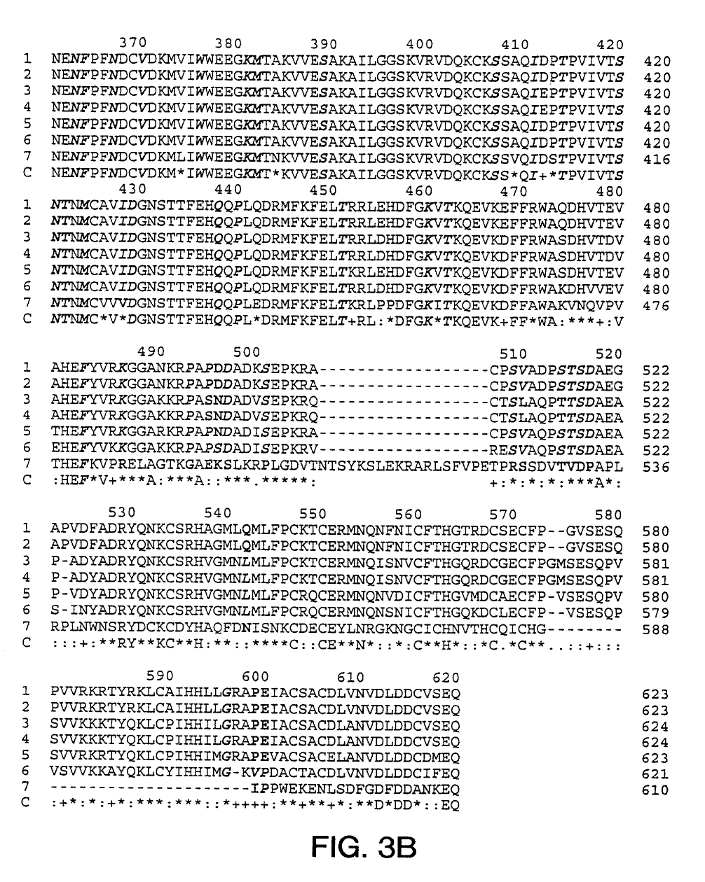

For the alignments presented herein (see, FIGS. 3A and 3B) for the AAV serotype, the CLUSTALW program was employed with parameters set as follows: scoring matrix BLOSUM, gap open 10, gap extend 0.1, gap distance 40% and transitions/transversions 0.5; specific residue penalties for hydrophobic amino acids (DEGKNPQRS), distance between gaps for which the penalties are augmented was 8, and gaps of extemeties penalized less than internal gaps.

As used herein, a “corresponding” position on a protein, such as the AAV rep protein, refers to an amino acid position based upon alignment to maximize sequence identity. For AAV Rep proteins an alignment of the Rep 78 protein from AAV-2 and the corresponding protein from other AAV serotypes (AAV-1, AAV-6, AAV-3, AAV-3B, AAV-4, AAV-2 and AAV-5) is shown in FIGS. 3A and 3B. The “hit” positions are shown in italics.

As used herein, the term at least “90% identical to” refers to percent identities from 90 to 100% relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, it is also understood that the terms substantially identical or similar varies with the context as understood by those skilled in the relevant art.

As used herein, genetic therapy involves the transfer of heterologous nucleic acids to the certain cells, target cells, of a mammal, particularly a human, with a disorder or conditions for which such therapy is sought. The nucleic acid, such as DNA, is introduced into the selected target cells in a manner such that the heterologous nucleic acid, such as DNA, is expressed and a therapeutic product encoded thereby is produced. Alternatively, the heterologous nucleic acid, such as DNA, may in some manner mediate expression of DNA that encodes the therapeutic product, or it may encode a product, such as a peptide or RNA that in some manner mediates, directly or indirectly, expression of a therapeutic product. Genetic therapy may also be used to deliver nucleic acid encoding a gene product that replaces a defective gene or supplements a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product may be modified prior to introduction into the cells of the afflicted host in order to enhance or otherwise alter the product or expression thereof. Genetic therapy may also involve delivery of an inhibitor or repressor or other modulator of gene expression.

As used herein, heterologous or foreign nucleic acid, such as DNA and RNA, are used interchangeably and refer to DNA or RNA that does not occur naturally as part of the genome in which it is present or which is found in a location or locations in the genome that differ from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is expressed. Any DNA or RNA that one of skill in the art would recognize or consider as heterologous or foreign to the cell in which it is expressed is herein encompassed by heterologous DNA. Heterologous DNA and RNA may also encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and DNA that encodes other types of proteins, such as antibodies.

Hence, herein heterologous DNA or foreign DNA, includes a DNA molecule not present in the exact orientation and position as the counterpart DNA molecule found in the genome. It may also refer to a DNA molecule from another organism or species (ie., exogenous).

As used herein, a therapeutically effective product introduced by genetic therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease.

As used herein, A therapeutically effective dose refers to that amount of the compound sufficient to result in amelioration of symptoms of disease.

As used herein, isolated with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compounds can be substantially purified by the one-step method described in Smith and Johnson, Gene 67:31-40 (1988). The terms isolated and purified are sometimes used interchangeably.

Thus, by “isolated” is meant that the nucleic is free of the coding sequences of those genes that, in the naturally-occurring genome of the organism (if any) immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, receptor refers to a biologically active molecule that specifically binds to (or with) other molecules. The term “receptor protein” may be used to more specifically indicate the proteinaceous nature of a specific receptor.

As used herein, recombinant refers to any progeny formed as the result of genetic engineering.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression a DNA sequence and a regulatory sequence(s) are connected in such a way to control or permit gene expression when the appropriate molecular, e.g., transcriptional activator proteins, are bound to the regulatory sequence(s).

As used herein, production by recombinant means by using recombinant DNA methods means the use of the well known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds. It may be a solution, a suspension, liquid, powder, a paste, aqueous, non-aqueous or any combination thereof.

As used herein, a combination refers to any association between two or more items.

As used herein, substantially identical to a product means sufficiently similar so that the property of interest is sufficiently unchanged so that the substantially identical product can be used in place of the product.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, vector is also used interchangeable with “virus vector” or “viral vector”. In this case, which will be clear from the context, the “vector” is not self-replicating. Viral vectors are engineered viruses that are operatively linked to exogenous genes to transfer (as vehicles or shuttles) the exogenous genes into cells.

As used herein, transduction refers to the process of gene transfer and expression into mammalian and other cells mediated by viruses. Transfection refers to the process when mediated by plasmids.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, referred to as a single nucleotide polymorphism (SNP), the identity of which differs in different alleles. A polymorphic region can also be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

As used herein, the term “gene” or “recombinant gene” refers to a nucleic acid molecule comprising an open reading frame and including at least one exon and (optionally) an intron sequence. A gene can be either RNA or DNA. Genes may include regions preceding and following the coding region (leader and trailer).

As used herein, “intron” refers to a DNA sequence present in a given gene which is spliced out during mRNA maturation. As used herein, “nucleotide sequence complementary to the nucleotide sequence set forth in SEQ ID NO: x” refers to the nucleotide sequence of the complementary strand of a nucleic acid strand having SEQ ID NO: x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO: x refers to the complementary strand of the strand having SEQ ID NO: x or to any nucleic acid having the nucleotide sequence of the complementary strand of SEQ ID NO: x. When referring to a single stranded nucleic acid having the nucleotide sequence SEQ ID NO: x, the complement of this nucleic acid is a nucleic acid having a nucleotide sequence which is complementary to that of SEQ ID NO: x.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, an array refers to a collection of elements, such as nucleic acid molecules, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interation of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which may be any shape, including random shapes, needles, fibers, and elongated. Roughly spherical “beads”, particularly microspheres that can be used in the liquid phase, are also contemplated. The “beads” may include additional components, such as magnetic or paramagnetic particles (see, e.g., Dyna beads (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and may be order of cubic microns. Such particles are collectively called “beads.”

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Directed Evolution of a Viral Gene

Recombinant viruses have been developed for use as gene therapy vectors. Gene therapy applications are hampered by the need for development of vectors with traits optimized for this application. The high throughput methods provided herein are ideally suited for development of such vectors. In addition to use for development of recombinant viral vectors for gene therapy, these methods can also be used to study and modify the viral vector backbone architechture, trans-complementing helper functions, where appropriate, regulatable and tissue specific promoters and transgene and genomic sequence analyses. Recombinant AAV (rAAV) is a gene therapy vector that can serve these and other purposes.

The rep protein is a adeno-associated virus protein involved in a number of biological processes necessary to AAV replication. The production of the rRep proteins enables viral DNA to replicate, encapsulate and integrate (McCarty et al. (1992) J. Virol. 66:4050-4057; Horer et al (1995) J. Virol. 69:5485-5496, Berns et al (1996) Biology of Adeno-associated virus, in Adeno-associated virus (AAV) Vectors in Gene Therapy, K. I. Berns and C. Giraud, Springer (1996); and Chlorini et al. (1996) The Roles of AAV Rep Proteins in gene Expression and Targeted Integration, from Adeno-associated virus (AAV) Vectors in Gene Therapy, K. I. Berns and C. Giraud, Springer (1996)). A rep protein with improved activity could lead to increased amounts of virus progeny thus allowing higher productivity of rAAV vectors.

Since the Rep protein is involved in replication it can serve as a target for increasing viral production. Since it has a variety of functions and its role in replication is complex, it has heretofore been difficult to identify mutations that result in increase viral production. The methods herein, which rely on in vivo screening methods, permit optimization of its activites as assessed by increases in viral production. Provided herein are Rep proteins and viruses and viral vectors containing the mutated Rep proteins that provide such increase. The amino acid positions on the rep proteins that are relevant for rep proteins activities in terms of AAV or rAAV virus production are provided. Those amino acid position are such that a change in the amino acid leads to a change in protein activity either to lower activity or increase activity. As shown herein, the alanine or amino acid scan revealed the amino acid positions important for such activity (i.e. hits). Subsequent mutations produced by systematically replacing the amino acids at the hit positions with the remaining 18 amino acids produced so-called “leads” that have amino acid changes and result in higher virus production. In this particular example, the method used included the following specific steps.

Amino Acid Scan

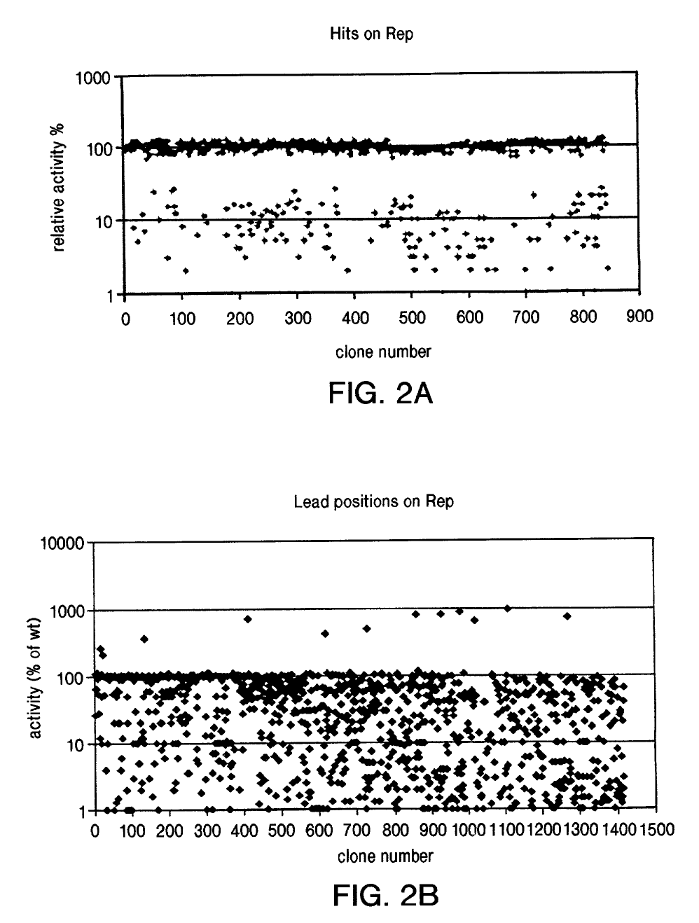

In order to first identify those amino acid (aa) positions on the rep protein that are involved in rep protein activity, an Ala-scan was performed on the rep sequence. For this, each aa in the rep protein sequence was individiually changed to Alanine. Any other amino acid, particularly another amino acid such as Gly or Ser that has a neutral effect on structure, could have been used. Each resulting mutant rep protein was then expressed and the amount of virus it produced was measured. The relative activity of each individual mutant compared to the native protein is indicated in FIG. 2A. HITS are those mutants that produce a decrease in the activity of the protein (in the example: all the mutants with activities below about 20% of the native activity).

In a second experimental round, which included a new set of mutations and phenotypic analysis, each amino acid position hit by the Ala-scan step, was mutated by amino acid replacement of the native amino acid by the remaining 18 amino acids, using site directed-mutagenesis.

In both rounds, each mutant was individually designed, generated and processed separately, and optionally in parallel with the other mutants. Neither combinatorial generation of mutants nor mixtures thereof were used in any step of the method.

A plasmid library was thus generated in which each plasmid contained a different mutant bearing a different amino acid at a different hit position. Again, each resulting mutant rep protein was then expressed and the amount of virus it could produced measure as indicated below. The relative activity of each individual mutant compared to the native protein is indicated in FIG. 2B. LEADS are those mutants that lead to an increase in the activity of the protein (in the example: the ten mutants with activities higher, typically between 2 to 10 times or more, generally 6-10 time, than the native activity).

Expression of the Genetic Variants and Phenotypic Characterization.

The rep protein acts as an intracellular protein through complex interaction with a molecular network composed by cellular proteins, DNA, AAV proteins and adenoviral proteins (note: some adenovirus proteins have to be present for the rep protein to work). The final outcome of the rep protein activity is the virus offspring composed by infectious rAAV particles. It can be expected that the activity of rep mutants would affect the titer of the rAAV virus coming out of the cells.

As the phenotypic characterization of the rep variants can only be accomplished by assaying its activity from inside mammalian cells, a mammalian cell-based expression system as well as a mammalian cell-based assay was used. The individual rep protein variants were expressed in human 293 HEK cells, by transfection of the individual plasmids constituting the diverse plasmid library. All necessary functions were provided as follows:

(a) the cellular proteins present in the permissive specific 293 HEK cells;

(b) the AAV necessary proteins and DNA were provided by co-transfection of the AAV cap gene as well as a rAAV plasmid vector providing the necessary signaling and substrate ITRs sequences;

(c) the adenovirus (AV) proteins were provided by co-transfection with a plasmid expressing all the AV helper functions.

A library of recombinant viruses with mutant rep encoding genes was generated. Each recombinant, upon introduction into a mammalian cell and expression resulted in production of rAAV infectious particles. The number of infectious particles produced by each recombinant was determined in order to assess the activity of the rep variant that had generated that amount of infectious particles.

The number of infectious particles produced was determined in a cell-based assay in which the activity of a reporter gene, in the exemplified embodiment, the bacterial lacZ gene, or virus replication (Real time PCR) was performed to quantitatively assess the number of viruses. The limiting dilution (titer) for each virus preparation (each coming from a different rep variant) was determined by serial dilution of the viruses produced, followed by infection of appropriate cells (293 HEK or HeLa rep/cap 32 cells) with each dilution for each virus and then by measurement of the activity of the reporter gene for each dilution of each virus. Hill plots (NAUTSCAN™) (published as International PCT application No. WO 01/44809 based on PCT n° PCT/FR00/03503, December, 2000; see EXAMPLES) or a second order polynomial function (Drittanti et al. (2000) Gene Ther. 7: 924-929; see co-pending U.S. provisional application Serial No. Attorney Dkt. No. 37851-P911) was used to analyze the readout data and to calculate the virus titers. Briefly, the titer was calculated from the second order polynomial function by non-linear regression fitting of the experimental data. The point where the polynomial curve reaches its minimum is considered to be the titer of the rAAV preparation. Results are shown in the EXAMPLE below.

Comparison Between Results of Full-Length Hit Position Analysis Reporter Here and the Literature

The experiments identified a number of heretofore unknown mutation loci, which include the hits at positions: 4, 20, 22, 28, 32, 38, 39, 54, 59, 124, 125, 127, 132, 140, 161, 163, 193, 196, 197, 221, 228, 231, 234, 258, 260, 263, 264, 334, 335, 341, 342, 347, 350, 354, 363, 364, 367, 370, 376, 381, 389, 407, 411, 414, 420, 421, 422, 428, 429, 438, 440, 451, 460, 462, 484, 488, 495, 497, 498, 499, 503, 511, 512, 516, 517 and 518 with reference to the amino acids in Rep78 and Rep 68. Rep 78 is encoded by nucleotides 321-2,186; Rep 68 is encoded by nucleotides 321-1906 and 2228-2252; Rep 52 is encoded by nucleotides 993-2186, and Rep 40 is encoded by amino acids 993-1906 and 2228-2252 of wildtype AAV.

Also among these are mutations that may have multiple effects. Since the Rep coding region is quite complex, some of the mutations have several effects. Amino acids 542, 598, 600 and 601, which are in the to the Rep 68 and 40 intron region, are also in the coding region of Rep 78 and 52. Codon 630 is in the coding region of Rep 68 and 40 and non coding region of Rep 78 and 52.

Mutations at 10, 86, 101, 334 and 519 have been previously identified, and mutations, at loci 64, 74, 88, 175, 237, 250 and 429, but with different amino acid substitutions, have been previously reported. In all instances, however, the known mutations reportedly decrease the activity of Rep proteins. Among mutations described herein, are mutations that result in increases in the activity the Rep function as assessed by detecting increased AAV production.

In particular, as described in the Example, mutations in the Rep-encoding region of AAV, including serotypes AAV-1, AAV-2, AAV-3, AAV-3B, AAV-4, AAV-5 and AAV-6 are provided (see Example below). The mutant proteins and mutant adeno-associate virus (AAV) Rep proteins are provided. Exemplary proteins with mutations at one or more of residues 4, 20, 22, 29, 32, 38, 39, 54, 59, 124, 125, 127, 132, 140, 161, 163, 193, 196, 197, 221, 228, 231, 234, 258, 260, 263, 264, 334, 335, 337, 342, 347, 350, 354, 363, 364, 367, 370, 376, 381, 389, 407, 411, 414, 420, 421, 422, 424, 428, 438, 440, 451, 460, 462, 484, 488, 495, 497, 498, 499, 503, 511, 512, 516, 517, 518, 542, 548, 598, 600 and 601 of AAV-2 or the corresponding residues in other serotypes. Residue 1 corresponds to residue 1 of the Rep78 protein encoded by nucleotides 321-323 of the AAV-2 genome (see FIG. 3 and the Table below for an alignment of the mutations from various serotypes).

Of particular interest are mutations that increase activity of the Rep proteins compared to wildtype. Such mutations include one or more of residues 350, 462, 497, 517, 542, 548, 598, 600 and 630 of AAV-2 and the corresponding residues in other serotypes. Also provided are mutations at or near those residues, such as within about 1 to about 10 residues of these residues such that the resulting protein has increased activity. Mutations include insertions, deletions and replacements.

Lead Identification.

Based on the results obtained from the assays described herein (i.e. titer of virus produced by each rep variant), each individual rep variant was assigned a specific activity. Those variant proteins displaying the highest titers were selected as leads and are used to produce rAAV.

In further steps, rAAV and Rep proteins that contain a plurality of mutations based on the hits (see Table in the EXAMPLE, listing the hits and lead sites), are produced to produce rAAV and Rep proteins that have activity that is further optimized. Examples of such proteins and AAV containing such proteins are described in the EXAMPLE. Other combinations of mutations can be prepared and tested as described herein to identify other leads of interest, particularly those that have increased Rep protein activity or that result in higher viral titers in cells containing such viruses that include appropriate cis acting elements for viral production.

The rAAV rep mutants are used as expression vectors, which, for example, can be used transiently for the production of recombinant AAV stocks. Alternatively, the recombinant plasmids may be used to generate stable packaging cell lines.

Also among the uses of rAAV, particularly the high titer stocks produced herein, is gene therapy for the purpose of transferring genetic information into appropriate host cells for the management and correction of human diseases including inherited and acquired disorders such as cancer and AIDS. The rAAV can be administered to a patient at therapeutically effective doses.

C. Uses of the Mutant Rep Genes and the rAAV Gene Therapy

The rAAV provided herein are intended for use as vectors for gene therapy. The rAAV provided herein are intended for use in any gene therapy protocol the uses AAV as a vector. The mutant Rep proteins and nucleic acid molecules can be used to replace the corresponding gene in other AAV vectors. Of interest are the mutations provided herein that increase rAAV production. In particular, the mutant Rep proteins are used to increase production of rAAV derived from any of the AAV seroptyes, including AAV-1, AAV-2, AAV-3, AAV-3B, AAV-4, AAV-5 and AAV-6 serotypes.

Toxicity and therapeutic efficacy of the rAAV can be determined by standard pharmaceutical procedures in cell cultures or experimental animals, e.g., for determining the LDS₅₀ (the dose lethal to 50% of the population) and the ED50 (the dose therapeutically effective in 50% of the population). The dose ratio between toxic and therapeutic effects is the therapeutic index and it can be expressed as the ratio LD₅₀/ED₅₀. Doses that exhibit large therapeutic indices are preferred. Doses that exhibit toxic side effects may be used, care should be taken to design a delivery system that targets rAAV to the site of treatment in order to minimize damage to untreated cells and reduce side effects.

The data obtained from cell culture assays and animal studies can be used in formulating a range of dosage for use in humans. The dosage of such rAAV lies preferably within a range of circulating concentrations that include the ED₅₀ with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC₅₀ (ie., the concentration of the test compound which achieves a half-maximal infection or a half-maximal inhibition) as determined in cell culture. Such information can be used to more accurately determine useful doses in humans. Levels in plasma may be measured, for example, by high performance liquid chromatography.

Treatment of Cancer, HIV, and Papilloma and Herpes Virus Infections and Diseases Mediated Thereby

AAV, which is a helper-dependent parvovirus requires co-infection with an adenovirus, herpes virus or papilloma virus (PV) for replication and particle formation. AAV inhibits PV-induced oncogenic tansformation, and this inhibition has been mapped to the Rep78 protein. The Rep78 protein ihibits expression of the PV promoter just upstream of the E6 gene (p89 of bovine PV-1 (BPV-1)) p97 of human PV-16 (HPV-16), and p105 of human PV-18 (HPV-18)). DNA binding is required for this inhibition. Rep78 also binds to the TAR sequences (nt+23 to +42) and to a region just upstream of the TATA box (nt. −54 to −34) in the HIV LTR region. AAV Rep78 also regulates a variety of other cancer associated genes, including, but are not limited to, C—H-ras (Khleif et al. (1991) Viology 181:738-741), c-fos and c-myc (Hermonat (1994) Cancer Lttrs 81:129-136);

Infection by AAV is negatively associated with cervical cancer. Infection and DNA integration by certain PV types are central events in the etiology of cervical cancer (Durst et al. (1983) Proc. Natl. Acad. Sci. U.S.A. 80:3812-3815; Cullen et al. (1991) J. Virol. 65:606-612). Roughly two thirds of cervical cancers contain the HPV-16 virus. AAV is also commonly found in the anogenital region (Han et al. (1996) Virus Genes 12:47-52.

Contemplated herein are AAV rep mutants that bind with greater than wild-type AAV Rep78 to nucleic acid from PV, AAV, oncogenes or HIV, particularly HIV-1, and particularly promoter and other transcriptional/translational regulatory sequences from these sources. The mutant Rep protein when administered to a subject can inhibit PV and PV-associated diseases, HIV and HIV-associated diseases. Hence methods for treatment of PV and HIV-mediated disorders by administration of rAAV encoding mutant the Rep78 genes are provided. The particular mutants for use in these methods can be identified by testing each mutant for inhibitory activity, for example, in cell-based assays. For example, the Rep mutant protein can be tested by contacting it with nucleic acid from a PV, AAV or HIV or oncogene for a time sufficient to permit binding thereto, and comparing such binding to the binding of a wild-type Rep protein under the same conditions. Alternatively competitive binding assays may be performed. Mutant proteins having higher binding affinities are identified.

Fusion proteins containing a tat protein of HIV or other targeting agent and mutant Rep protein are also provided. Pharmaceutical compositions containing such fusion proteins are provided. The fusion proteins can contain additional components, such as E. coli maltose binding protein (MBP) that aid in uptake of the protein by cells (see, International PCT application No. WO 01/32711). Nucleic acid molecules encoding the mtuant Rep protein or fusion protein operably linked to a promoter, such as an inducible promoter for expression in mammalian cells are also provided. Such promoters include, but are not limited to, CMV and SV40 promoters; adenovirus promoters, such as the E2 gene promoter, which is responsive to the HPV E7 oncoprotein; a PV promoter, such as the PBV p89 promoter that is responsive to the PV E2 protein; and other promoters that are activated by the HIV or PV or oncogenes.

The mutant rep proteins are also delivered to the cells in rAAV or a portion thereof that can additionally encoded therapeutic agents for treatment of the cancer or HIV infection or other disorder.

Methods of inhibiting oncogenic transformation by bovine PV (BPV) and by human PV (HPV) are provided.

Methods of inhibiting PV, PV-associated diseases, HIV and HIV-associated diseases are provided. These methods are practiced by administering the proteins, nucleic acids or rAAV or portions thereof to a subject, such as a mammal, including a human to thereby inhibit or modulate disease progression or oncogenic transformation.

Other Systems

It has been shown that the Rep protein can is involved in the regulation of gene expression, including viral replication as described above, cellular pathways and protein phosphorylation (see, e.g., Chlorini et al. (1998) Mol. Cell Biol. 18:5921-5929). Hence the mutant Rep proteins provided herein can be used to block, stimulate, inhibit, regulate or otherwise modulate metabolic or cellular signaling pathyways. Rep proteins provided herein can be used to block, stimulate, inhibit, regulate or otherwise modulate cyclic AMP response pathways, and also to regulate or modulate cellular promoters as a means of modulating gene expression. Methods using these proteins for such purposes are provided herein.

Formulation of rAAV

Pharmaceutical compositions containing the rAAV, fusion proteins or encoding nucleic acid molecules can beformulated in any conventional manner by mixing an a selected amount of rAAV with one or more physiologically acceptable carriers or excipients. For example, the rAAV may be suspended in a carrier such as PBS (phosphate buffered saline). The active compounds can be administered by any appropriate route, for example, orally, parenterally, intravenously, intradermally, subcutaneously, or topically, in liquid, semi-liquid or solid form and are formulated in a manner suitable for each route of administration. Preferred modes of administration include oral and parenteral modes of administration.

The rAAV and physiologically acceptable salts and solvates may be formulated for administration by inhalation or insufflation (either through the mouth or the nose) or for oral, buccal, parenteral or rectal administration. For administration by inhalation, the rAAV can be delivered in the form of an aerosol spray presentation from pressurized packs or a nebulizer, with the use of a suitable propellant, e.g. dichlorodifluoromethane, trichlorofluoromethane, dichlorotetra-fluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a therapeutic compound and a suitable powder base such as lactose or starch.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

The rAAV may be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder lyophilized form for constitution with a suitable vehicle, e.g., sterile pyrogen-free water, before use.

In addition to the formulations described previously, the rAAV may also be formulated as a depot preparation. Such long acting formulations may be administered by implantation (for example, subcutaneously or intramuscularly) or by intramuscular injection. Thus, for example, the therapeutic compounds may be formulated with suitable polymeric or hydrophobic materials (for example as an emulsion in an acceptable oil) or ion exchange resins, or as sparingly soluble derivatives, for example, as a sparingly soluble salt.

The active agents may be formulated for local or topical application, such as for topical application to the skin and mucous membranes, such as in the eye, in the form of gels, creams, and lotions and for application to the eye or for intracisternal or intraspinal application. Such solutions, particularly those intended for ophthalmic use, may be formulated as 0.01%-10% isotonic solutions, pH about 5-7, with appropriate salts. The compounds may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma).

The concentration of active compound in the drug composition will depend on absorption, inactivation and excretion rates of the active compound, the dosage schedule, and amount administered as well as other factors known to those of skill in the art. For example, the amount that is delivered is sufficient to treat the symptoms of hypertension.

The compositions may, if desired, be presented in a pack or dispenser device which may contain one or more unit dosage forms containing the active ingredient. The pack may for example, comprise metal or plastic foil, such as a blister pack. The pack or dispenser device may be accompanied by instructions for administration.

The active agents may be packaged as articles of manufacture containing packaging material, an agent provided herein, and a label that indicates the disorder for which the agent is provided.

The following examples are included for illustrative purposes only and are not intended to limit the scope of the invention. The specific methods exemplified can be practiced with other species. The examples are intended to exemplify generic processes.

### EXAMPLE

Materials and Methods

Cells:

293 human embryo kidney (HEK) cells, obtained from ATCC, were cultured in Dulbecco's modified Eagle's medium containing 4.5 g/l glucose (DMEM; GIBGO-BRL) 10% fetal bovine serum (FBS, Hyclone). Hela rep-cap 32 cells, described above, were obtained from Anna Salvetti (CHU, Nantes) and cultured in the medium described above.

Plasmids:

pNB-Adeno, which encodes the entire E2A and E4 regions and VA RNA I and II genes of Adenovirus type 5, was constructed by ligating into the polylinker of multiple cloning site of pBSII KS (+/−) (Stratagene, San Diego, USA) the SalI-HindIII fragemnt (9842-11555 nt) of Adenovirus type 5) and the BamHI-ClaI fragment (21563-35950) of pBR325. All fragments of adenovirus gene were obtained from the plasmid pBHG-10 (Microbix, Ontario, Canada). pNB-AAV encodes the genes rep and cap of AAV-2 was constructing by ligation of XbaI-XbaI PCR fragment containing the genome of AAV-2 from nucleotide 200 to 4480 into XbaI site of polylinker MCS of pBSIIKS(+/−). The PCR fragment was obtained from pAV1 (ATCC, USA). Plasmid pNB-AAV was derived from plasmid pVA1I, which contains the AAV genomic region, rep and cap. pNB-AAV does not contain the AAV ITR's present in pAV1. pAAV-CMV(nls)LacZ was provided by Dr Anna Salvetti (CHU, Nantes).

Plasmid pCMV(nls)LacZ (rAAV vector plasmid) and pNB-Adeno were prepared on DH5a E. coli and purified by Nucleobond AX PC500 Kit (Macherey-Nagel), according to standard procedures. Plasmid pAAV-CMV(nls)LacZ is derived fom plasmid psub201 by deleting the rep-cap region with SnaB I and replacing it with an expression cassette harboring the cytomegalovirus (CMV) immediate early promoter (407 bp), the nuclear localized β-galactosidase gene and the bovine growth hormone polyA signal (324 bp) (see, Chadeuf et al. (2000) J. Gene Med. 2:260-268. pAAV-CMV(nls)LacZ was provided by Dr Anna Salvetti.

Virus:

Wild type adenovirus (AV) type 5 stock, originally provided by Dr Philippe Moullier (CHU, Nantes), was produced accordingly to standard procedures.

Construction of Rep Mutant Libraries

25 pmol of each mutagenic primer was placed into a 96 PCR well plate. 15 μl of reaction mix (0.25 pmol of pNB-AAV), 25 pmol of the selection primer (changing one non-essential unique restriction site to a new restriction site), 2 μl of 10× mutagenesis buffer (100 mM Tris-acetate pH 7.5, 100 mM MgOAc and 500 mM KOAc pH 7.5) was added into each well. The samples were incubated at 98° C. for 5 minutes and then immediately incubated for 5 minutes on ice. Finally, the plate was placed at room temperature for 30 minutes.

The primer extension and ligation reactions of the new strands were completed by adding to each sample: 7 μl of nucleotide mix (2.86 mM each nucleotide and 1.43× mutagenesis buffer) and 3 μl of a fresh 1:10 enzyme dilution mix (0.025 U/μl of native T7 DNA polymerase and 1 U/μl of T4 DNA ligase were diluted in 20 mM Tris HCl pH 7.5, 10 mM KCl, 10 mM β-mercaptoethanol, 1 mM DTT, 0.1 mM EDTA and 50% glycerol). Samples were incubated at 37° C. for 1 hour. The T4 DNA ligase was inactivated by incubating the reactions at 72° C. for 15 minutes to prevent re-ligation of the digested strands during the digestion of the parental plasmid (pNB-AAV).

Each mutagenesis reaction was digested with restriction enzyme to eliminate parental plasmids: 30 μl solution containing 3 μl of 10× enzyme digestion buffer and 10 units of restriction enzyme were added to each mutagenesis reaction and incubated at 37° C. for at least 3 hours.

90 μl of the E. coli XLmutS competent cells (Stratagene, San Diego Calif.; supplemented with 1.5 μl of β-mercaptoethanol to a final concentration of 25 mM) were aliquoted into prechilled deep-well plates. The plates were incubated on ice for 10 minutes and swirling gently every 2 minutes.

A fraction of the reactions that had been digested with restriction enzyme (1/10 of the total volume) was added to the deep well plates. The plates were swirled gently prior to incubation on ice for 30 minutes. A heat pulse was performed in a 42° C. water bath for 45 seconds, the transformation mixture was incubated on ice for 2 minutes and 0.45 ml of preheated SOC medium (2% (w/v) tryptone, 0.5% (w/v) yeast extract, 8.5 mM NaCl, 2.5 mM KCl, 10 mM MgCl₂ and 20 mM glucose at pH 7) was added. The plates were incubated at 37° C. for 1 hour with shaking.

To enrich for mutant plasmids, 1 ml of 2×YT broth medium (YT medium is 0.5% yeast extract, 0.5% NaCl, 0.8% bacto-tryptone), supplemented with 100 μg/ml of ampicillin, was added to each transformation mixture and the cultures were grown overnight at 37° C. with shaking. Plasmid DNA isolation was performed from each mutant culture using standard procedure described in Nucleospin Multi-96 Plus Plasmid Kit (Macherey-Nagel). Five hundred μg of the resulting isolated DNA was digested with 10 units of the selection restriction enzyme in a total volume of 30 μl containing 3 μl of 10×enzyme digestion buffer for overnight at 37° C.

A fraction of the digested reactions (1/10 of the total volume) were transformed into 40 μl of Epicurian coli XL1-Blue competent cells supplemented with 0.68 μl of β-mercaptoethanol to a final concentration of 25 mM. After heat pulse, 0.45 ml of SOC was added and the transformation mixtures were incubated for 1 hour at 37° C. with shaking before to be plate on LB-ampicillin agar plates. The agar plates were incubated overnight at 37° C. and the colonies obtained were picked up and grown overnight at 37° C. into deep-well plates.

Four clones per reaction were screened for the presence of the mutation using restriction enzyme specific to the new restriction site introduced into the mutated plasmid with the selection primer. The cDNA from selected clones was also sequenced to confirm the presence of the expected mutation.

Monitoring rAAV Production

rAAV from each of the above wells, were produced by triple transfection on 293 HEK cells. 3×10⁴ cells were seeded into each well of 96 micro-well plate and cultured for 24 hours before transfection. Transfection was made on cells at about 70% confluenacy. 25 kDa PEI (poly-ethylene-imine, Sigma-Aldrich) was used for the triple transfection step. Equimolar amounts of the three plasmids AV helper plasmid (pNB-Adeno), AAV helper plasmid (pNB-AAV or a mutant clone rep plasmid) and vector plasmid (pAAV-CMV(nls)LacZ) were mixed with 10 mM PEI by gently shaking. The mixture was the added to the medium culture on the cells. 60 hours after transfection, the culture medium was replaced with 100 μl of lysis buffer (50 mM Hepes, pH 7.4; 150 mM NaCl; 1 mM MgCl₂; 1 mM CaCl₂; 0.01% CHAPS). After one cycle of freeze-thawing the cellular lysate was filtered through a millipore filter 96 well plate and stored at −80° C.

rAAV Infection Particles (ip)

Titers of rAAV vector particles were determined on HeLa rep/cap 32 cells using standard dRA (serial dilution replication assay) test. Cells were plated 24 hours before infection at a density of 1×10⁴ cells in 96-well plates. Serial dilutions of the rAAV preparation were made between 1 and 1×10⁶ μl and used for co-infection of the HeLa rep/cap 32 cells together with wt-AV type 5 (MOI 25). 48 hours after infection the ip were measured by real time PCR or by the quantification of biological activity of the transgene.

Real Time PCR

Infected HeLa rep/cap 32 cells were lysed with 50 μl of solution (50 mM Hepes, pH 7.4; 150 mM NaCl). After one cycle of freeze-thawing 50 μl of Proteinase K (10 mg/ml) and the lysate were incubated one hour at 55° C. The enzyme was inactivated by incubation 10 min at 96° C.

For real time PCR, 0.2 μl of lysate was taken. Final volume of the reaction was 10 μl in 384 well plate using an Applied Biosystem Prism 7900. The primers and fluorescence probe set corresponding to the CMV promoter were as follows: CMV 1 primer 5′-TGCCAAGTACGCCCCCTAT-3′ (SEQ ID No. 733) (0.2 μM) and CMV 2 primer 5′-AGGTCATGTACTGGGCATAATGC-3′ (SEQ ID No. 734) (0.2 μM); probe VIC-Tamra 5′-TCAATGACGGTAAATGGCCCGCCT-3′ (SEQ ID No. 735) (0.1 μM). dRA plots were obtained by plotting the DNA copy number (obtained by real time PCR) vs. the dilution of the rAAV preparation.

β-Galactosidase Activity

After 48 hours of infection, cells were treated with trypsine, and 100 μl of reaction solution (GalScreen Kit, Tropix) was added and incubated for one hour at 26° C. Luminescence was measured in NorthStar (Tropix) HTS station. dRA plots were obtained plotting the intensity of β-Galatosidase activity vs. the dilution of the rAAV preparation.

Mathematical Model for Results Analysis:

Results were analyzed using the Hill equation-based analysis (designated NautScan™; see, Patent n° 9915884, 1999, France; published as International PCT application No. WO 01/44809 (PCT n° PCT/FR00/03503, Dec, 2000). Briefly, data were processed using a Hill equation-based model that allows extraction of key feature indicators of performance for each individual mutant. Mutants were ranked based on the values of their individual performance and those at the top of the ranking list were selected as Leads.

Results

Generation of Diversity.

To identify candidate amino acid (aa) positions on the rep protein involved in rep protein activity an Ala-scan was performed on the rep sequence. For this, each amino acid in the rep protein sequence was replaced with Alanine. To do this sets of rAAV that encode mutant rep proteins in which each differs from wild type by replacement of one amino acid with Ala, was generated. Each set of rAAV was individually introduced into cells in a well of a microtiter plate, under conditions for expression of the rep protein. The amount of virus that could be produced from each variant was measured as described below. Briefly, activity of Rep was assessed by determining the amount of AAV or rAAV produced using infection assays on HeLa Rep-cap 32 cells and by measurement of AAV DNA replication using Real Time PCR, or by assessing transgene (β-galactosidase) expression. The relative activity of each individual mutant compared to the native protein was assessed and “hits” identified. Hit positions are the positions in the mutant proteins that resulted in an alteration (selected to be at least about 20%), in this instance all resulted in a decrease, in the amount of virus produced compared to the activity of the native (wildtype) gene (see FIG. 2A).

The hits were then used for identification of leads (see, FIG. 2B). Assays for Rep activity were performed as described for identification of the hit positions. Hit positions on Rep proteins and the effect of specific amino acids on the productivity of AAV-2 summarized in the following table:

| Hit position | replacing amino acid (effect) | |
|---|---|---|
| 4 (ttt) F | (gct) A (decrease) | |
| 10 (aag) K | (gcg) A (decrease) | |
| 20 (ccc) P | (gcc) A (decrease) | |
| 22 (att) I | (gct) A (decrease) | |
| 28 (tgg) W | (gcg) A (decrease) | |
| 32 (gag) E | (gcg) A (decrease) | |
| 38 (ccg) P | (gcg) A (decrease) | |
| 39 (cca) P | (gca) A (decrease) | |
| 54 (ctg) L | (gct) A (decrease) | |
| 59 (ctg) L | (gcg) A (decrease) | |
| 64 (ctg) L | (gcg) A (decrease) | |
| 74 (ccg) P | (gcg) A (decrease) | |
| 86 (gag) E | (gcg) A (decrease) | |
| 88 (tac) Y | (gcc) A (decease) | |
| 101 (aaa) K | (gca) A (decrease) | |
| 124 (atc) I | (gcc) A (decrease) | |
| 125 (gag) E | (gcg) A (decrease) | |
| 127 (act) T | (gct) A (decrease) | |
| 132 (ttc) F | (gcc) A (decrease) | |
| 140 (ggc) G | (gcc) A (decrease) | |
| 161 (acc) T | (gcc) A (decrease) | |
| 163 (cct) P | (gct) A (decrease) | |
| 175 (tat) Y | (gct) A (decrease) | |
| 193 (ctg) L | (gcg) A (decrease) | |
| 196 (gtg) V | (gcg) A (decrease) | |
| 197 (tcg) S | (gcc) A (decrease) | |
| 221 (tca) S | (gca) A (decrease) | |
| 228 (gtc) V | (gcg) A (decrease) | |
| 231 (ctc) L | (gcc) A (decrease) | |
| 234 (aag) K | (gcg) A (decrease) | |
| 237 (acc) T | (gcc) A (decrease) | |
| 250 (tac) Y | (gcc) A (decrease) | |
| 258 (aac) N | (gcc) A (decrease) | |
| 260 (cgg) R | (gcg) A (decrease) | |
| 263 (atc) I | (gcc) A (decrease) | |
| 264 (aag) K | (gcg) A (decrease) | |
| 334 (ggg) G | (gcg) A (decrease) | |
| 335 (cct) V | (gct) A (decrease) | |
| 337 (act) T | (gct) A (decrease) | |
| 341 (acc) T | (gcc) A (decrease) | |
| 342 (aac) N | (gcc) A (decrease) | |
| 347 (ata) I | (gca) A (decrease) | |
| 350 (act) T | (gct) A (decrease | (aat) N (increase) |
| 354 (tac) Y | (gcc) A (decrease) | |
| 363 (aac) N | (gcc) A (decrease) | |
| 364 (ttt) F | (gct) A (decrease) | |
| 367 (aac) N | (gcc) A (decrease) | |
| 370 (gtc) V | (gcc) A (decrease) | |
| 376 (tgg) W | (gcg) A (decrease) | |
| 381 (aag) K | (gcg) A (decrease) | |
| 382 (atg) M | (gcg) A (decrease) | |
| 389 (tcg) S | (gcg) A (decrease) | |
| 407 (tcc) S | (gcc) A (decrease) | |
| 411 (ata) I | (gca) A (decrease) | |
| 414 (act) T | (gct) A (decrease) | |
| 420 (tcc) S | (gct) A (decrease) | |
| 421 (aac) N | (gcc) A (decrease) | |
| 422 (acc) T | (gcc) A (decrease) | |
| 424 (atg) M | (gcg) A (decrease) | |
| 428 (att) I | (gct) A (decrease) | |
| 429 (gac) D | (gcc) A (decrease) | |
| 438 (cag) Q | (gcg) A (decrease) | |
| 440 (ccg) P | (gcg) A (decrease) | |
| 451 (acc) T | (gcc) A (decrease) | |
| 460 (aag) K | (gcg) A (decrease) | |
| 462 (acc) T | (gcc) A (decrease) | (ata) I (increase) |
| 484 (ttc) F | (gcc) A (decrease) | |
| 488 (aag) K | (gcg) A (decrease) | |
| 495 (ccc) P | (gcc) A (decrease) | |
| 497 (ccc) P | (gcc) A (decrease) | (cga) R (increase) |
| 497 (ccc) P | (gcc) A (decrease) | (ctc) L (increase) |
| 497 (ccc) P | (gcc) A (decrease) | (tac) Y (increase) |
| 498 (agt) S | (gct) A (decrease) | |
| 499 (gac) D | (gcc) A (decrease) | |
| 503 (agt) S | (gcg) A (decrease) | |
| 511 (tca) S | (gca) A (decrease) | |
| 512 (gtt) V | (gct) A (decrease) | |
| 516 (tcg) S | (gcg) A (decrease) | |
| 517 (acg) T | (gct) A (decrease) | (aac) N (increase) |
| 518 (tca) S | (gca) A (decrease) | |
| 519 (gac) D | (gcg) A (decrease) | |
| 542 (ctg) L | (gcg) A (decrease) | (tcg) S (increase) |
| 548 (aga) R | (gca) A (decrease) | (agc) S (increase) |
| 598 (gga) G | (gca) A (decrease) | (agc) S (increase) |
| 600 (gtg) V | (gcg) A (decrease) | (ccg) P (increase) |
| 601 (cca) P | (gca) A (decrease) | |
| Hit position | | |
| (within intron) | replacing sequence (effect) | |
| 630 (tgc) | gcg (decrease) | cgc or tca or cct |
| | | (increase) |

The hits in other AAV serotypes (see, also FIGS. 3A and 3B) are as follows:

HIT POSITION

| AAV-2 | AAV-1 | AAV-3 | AAV-3B | AAV-4 | AAV-6 | AAV-5 |
|---|---|---|---|---|---|---|
| 4 | 4 | 4 | 4 | 4 | 4 | 4 |
| 10 | 10 | 10 | 10 | 10 | 10 | 10 |
| 20 | 20 | 20 | 20 | 20 | 20 | 20 |
| 22 | 22 | 22 | 22 | 22 | 22 | 22 |
| 29 | 29 | 29 | 29 | 29 | 29 | 29 |
| 32 | 32 | 32 | 32 | 32 | 32 | 32 |
| 38 | 38 | 38 | 38 | 38 | 38 | 38 |
| 39 | 39 | 39 | 39 | 39 | 39 | 39 |
| 54 | 54 | 54 | 54 | 54 | 54 | 54 |
| 59 | 59 | 59 | 59 | 59 | 59 | 59 |
| 64 | 64 | 64 | 64 | 64 | 64 | 64 |
| 74 | 74 | 74 | 74 | 74 | 74 | |
| 86 | 86 | 86 | 86 | 86 | 86 | 85 |
| 88 | 88 | 88 | 88 | 88 | 88 | 87 |
| 101 | 101 | 101 | 101 | 101 | 101 | 100 |
| 124 | 124 | 124 | 124 | 124 | 124 | 123 |
| 125 | 125 | 125 | 125 | 125 | 125 | 124 |
| 127 | 127 | 127 | 127 | 127 | 127 | 126 |
| 132 | 132 | 132 | 132 | 132 | 132 | 131 |
| 140 | 140 | 140 | 140 | 140 | 140 | |
| 161 | 161 | 161 | 161 | 161 | 161 | 158 |
| 163 | 163 | 163 | 163 | 163 | 163 | 160 |
| 175 | 175 | 175 | 175 | 175 | 175 | 172 |
| 193 | 193 | 193 | 193 | 193 | 193 | 190 |
| 196 | 196 | 196 | 196 | 196 | 196 | 193 |
| 197 | 197 | 197 | 197 | 197 | 197 | 194 |
| 221 | 221 | 221 | 221 | 221 | 221 | 217 |
| 228 | 228 | 228 | 228 | 228 | 228 | 224 |
| 231 | 231 | 231 | 231 | 231 | 231 | 227 |
| 234 | 234 | 234 | 234 | 234 | 234 | 230 |
| 237 | 237 | 237 | 237 | 237 | 237 | 233 |
| 250 | 250 | 250 | 250 | 250 | 250 | 246 |
| 258 | 258 | 258 | 258 | 258 | 258 | 254 |
| 260 | 260 | 260 | 260 | 260 | 260 | 256 |
| 263 | 263 | 263 | 263 | 263 | 263 | 259 |
| 264 | 264 | 264 | 264 | 264 | 264 | 260 |
| 334 | 334 | 334 | 334 | 334 | 334 | 330 |
| 335 | 335 | 335 | 335 | 335 | 335 | 331 |
| 337 | 337 | 337 | 337 | 337 | 337 | 333 |
| 341 | 341 | 341 | 341 | 341 | 341 | 337 |
| 342 | 342 | 342 | 342 | 342 | 342 | 338 |
| 347 | 347 | 347 | 347 | 347 | 347 | 342 |
| 350 | 350 | 350 | 350 | 350 | 350 | 346 |
| 354 | 354 | 354 | 354 | 354 | 354 | 350 |
| 363 | 363 | 363 | 363 | 363 | 363 | 359 |
| 364 | 364 | 364 | 364 | 364 | 364 | 360 |
| 367 | 367 | 367 | 367 | 367 | 367 | 363 |
| 370 | 370 | 370 | 370 | 370 | 370 | 366 |
| 376 | 376 | 376 | 376 | 376 | 376 | 372 |
| 381 | 381 | 381 | 381 | 381 | 381 | 377 |
| 382 | 382 | 382 | 382 | 382 | 382 | 378 |
| 389 | 389 | 389 | 389 | 389 | 389 | 385 |
| 407 | 407 | 407 | 407 | 407 | 407 | 403 |
| 411 | 411 | 411 | 411 | 411 | 411 | 407 |
| 414 | 414 | 414 | 414 | 414 | 414 | 410 |
| 420 | 420 | 420 | 420 | 420 | 420 | 416 |
| 421 | 421 | 421 | 421 | 421 | 421 | 417 |
| 422 | 422 | 422 | 422 | 422 | 422 | 418 |
| 424 | 424 | 424 | 424 | 424 | 424 | 420 |
| 428 | 428 | 428 | 428 | 428 | 428 | 424 |
| 429 | 429 | 429 | 429 | 429 | 429 | 425 |
| 438 | 438 | 438 | 438 | 438 | 438 | 434 |
| 440 | 440 | 440 | 440 | 440 | 440 | 436 |
| 451 | 451 | 451 | 451 | 451 | 451 | 447 |
| 460 | 460 | 460 | 460 | 460 | 460 | 456 |
| 462 | 462 | 462 | 462 | 462 | 462 | 458 |
| 484 | 484 | 484 | 484 | 484 | 484 | 480 |
| 488 | 488 | 488 | 488 | 488 | 488 | 484 |
| 495 | 495 | 495 | 495 | 495 | 495 | 491 |
| 497 | 497 | 497 | 497 | 497 | 497 | 493 |
| 498 | 498 | 498 | 498 | 498 | 498 | 494 |
| 499 | 499 | 499 | 499 | 499 | 499 | 495 |
| 503 | 503 | 503 | 503 | 503 | 503 | 499 |
| 511 | 511 | 511 | 511 | 511 | 511 | 529 |
| 512 | 512 | 512 | 512 | 512 | 512 | 530 |
| 516 | 516 | 516 | 516 | 516 | 516 | 534 |
| 517 | 517 | 517 | 517 | 517 | 517 | 535 |
| 518 | 518 | 518 | 518 | 518 | 518 | 536 |
| 519 | 519 | 519 | 519 | 519 | 519 | 537 |
| 542 | 543 | 542 | 542 | 542 | 543 | 561 |
| 548 | 549 | 548 | 548 | 548 | 549 | 567 |
| 598 | 599 | 600 | 600 | 599 | 599 | — |
| 600 | 602 | 603 | 603 | 602 | 602 | 589 |
| 601 | 603 | 604 | 604 | 603 | 603 | 590 |

Sets of nucleic acids encoding the rep protein were generated. The rep proteins encoded by these sets of nucleic acid molecules were those in which each amino acid position identied as a “hit” in the ala-scan step, were each sequentially replaced by all remaining 18 amino acids using site directed mutagenesis. Each mutant was designed, generated, processed and analyzed physically separated from the others in addressable arrays. No mixtures, pools, nor combinatorial processing were used.

As in the first round (alanine scan), a library of mutant rAAV was generated in which each individual mutant was independently and individually generated in a independent reaction and such that each mutant contains only a single amino acid change and this for each amino acid residue. Again, each resulting mutant rep protein was then expressed and the amount of virus produced in cells assessed and compared to the native protein.

Lead Identification

Since rep proteins that result in increased virus production are of interest, those mutants that lead to an increase in the amount of virus produced (2 to 10 times the native activity), were selected as “leads.”Ten such mutants were identified.

Based on the results obtained from the assays described above (i.e. titer of virus produced by each rep variant), each individual rep variant was assigned a specific activity. Those variant proteins displaying the highest titers were selected as leads (see Table above). Leads include: amino acid replacement of T by N at Hit position 350; T by I at Hit position 462; P by R at Hit position 497; P by L at Hit position 497; P by Y at Hit position 497; T by N at Hit position 517; L by S at Hit position 542; R by S at Hit positio 547, G by S at Hit position 598; G by D at Hit position 598; V by P at Hit position 600.

Also provided are combinations of the above mutant Rep 78, 68, 52. 40 proteins, nucleic acids encoding the proteins, and recombinant AAV (any serotype) contains the mutation at the indicated position or corresponding position for serotypes other than AAV-2, including any set forth in the following table and corresponding SEQ ID Nos. Each amino acid sequence is set forth in a separate sequence ID listing; for each mutation or combination thereof there is a single SEQ ID setting forth the unspliced nucleic acid sequence for Rep78/68, which for all mutations from amino acid 228 on, includes the corresponding Rep 52 and Rep 40 encoding sequence as well. Amino acid sequences of exemplary mutant Rep proteins

| | | | |
|---|---|---|---|
| Seq no. | gene | position(s) | codon(s) |

| | | | | |
|---|---|---|---|---|
| seq.1 | rep78 | 4 | GCT | |
| seq.2 | rep68 | 4 | GCT | |
| seq.3 | rep78 | 10 | GCG | |
| seq.4 | rep68 | 10 | GGG | |
| seq.5 | rep78 | 20 | GCC | |
| seq.6 | rep68 | 20 | GCC | |
| seq.7 | rep78 | 22 | GCT | |
| seq.8 | rep68 | 22 | GCT | |
| seq.9 | rep78 | 29 | GCG | |
| seq.10 | rep68 | 29 | GCG | |
| seq.11 | rep78 | 38 | GCG | |
| seq.12 | rep68 | 38 | GCG | |
| seq.13 | rep78 | 39 | GCA | |
| seq.14 | rep68 | 39 | GCA | |
| seq.15 | rep78 | 53 | GGT | |
| seq.16 | rep68 | 53 | GCT | |
| seq.17 | rep78 | 59 | GCG | |
| seq.18 | rep68 | 59 | GCG | |
| seq.19 | rep78 | 64 | GCT | |
| seq.20 | rep68 | 64 | GCT | |
| seq.21 | rep78 | 74 | GCG | |
| seq.22 | rep68 | 74 | GCG | |
| seq.23 | rep78 | 86 | GCG | |
| seq.24 | rep68 | 86 | GCG | |
| seq.25 | rep78 | 88 | GCC | |
| seq.26 | rep68 | 88 | GCC | |
| seq.27 | rep78 | 101 | GCA | |
| seq.28 | rep68 | 101 | GCA | |
| seq.29 | rep78 | 124 | GCC | |
| seq.30 | rep68 | 124 | GCC | |
| seq.31 | rep78 | 125 | GCG | |
| seq.32 | rep68 | 125 | GCG | |
| seq.33 | rep78 | 127 | GCT | |
| seq.34 | rep68 | 127 | GCT | |
| seq.35 | rep78 | 132 | GCC | |
| seq.36 | rep68 | 132 | GCC | |
| seq.37 | rep78 | 140 | GCC | |
| seq.38 | rep68 | 140 | GCC | |
| seq.39 | rep78 | 161 | GCC | |
| seq.40 | rep68 | 161 | GCC | |
| seq.41 | rep78 | 163 | GCT | |
| seq.42 | rep68 | 163 | GCT | |
| seq.43 | rep78 | 175 | GCT | |
| seq.44 | rep68 | 175 | GCT | |
| seq.45 | rep78 | 193 | GCG | |
| seq.46 | rep68 | 193 | GCG | |
| seq.47 | rep78 | 196 | GCC | |
| seq.48 | rep68 | 196 | GCC | |
| seq.49 | rep78 | 197 | GCC | |
| seq.50 | rep68 | 197 | GCC | |
| seq.51 | rep78 | 221 | GCA | |
| seq.52 | rep68 | 221 | GCA | |
| seq.53 | rep78 | 228 | GCG | |
| seq.54 | rep52 | 228 | GCG | |
| seq.55 | rep68 | 228 | GCG | |
| seq.56 | rep40 | 228 | GCG | |
| seq.57 | rep78 | 231 | GCC | |
| seq.58 | rep52 | 231 | GCC | |
| seq.59 | rep68 | 231 | GCC | |
| seq.60 | rep40 | 231 | GCC | |
| seq.61 | rep78 | 234 | GCG | |
| seq.62 | rep52 | 234 | GCG | |
| seq.63 | rep68 | 234 | GCG | |
| seq.64 | rep40 | 234 | GCG | |
| seq.65 | rep78 | 237 | GCC | |
| seq.66 | rep52 | 237 | GCC | |
| seq.67 | rep68 | 237 | GCC | |
| seq.68 | rep40 | 237 | GCC | |
| seq.69 | rep78 | 250 | GCC | |
| seq.70 | rep52 | 250 | GCC | |
| seq.71 | rep68 | 250 | GCC | |
| seq.72 | rep40 | 250 | GCC | |
| seq.73 | rep78 | 258 | GCC | |
| seq.74 | rep52 | 258 | GCC | |
| seq.75 | rep68 | 258 | GCC | |
| seq.76 | rep40 | 258 | GCC | |
| seq.77 | rep78 | 260 | GCG | |
| seq.78 | rep52 | 260 | GCG | |
| seq.79 | rep68 | 260 | GCG | |
| seq.80 | rep40 | 260 | GCG | |
| seq.81 | rep78 | 263 | GCC | |
| seq.82 | rep52 | 263 | GCC | |
| seq.83 | rep68 | 263 | GCC | |
| seq.84 | rep40 | 263 | GCC | |
| seq.85 | rep78 | 264 | GCG | |
| seq.86 | rep52 | 264 | GCG | |
| seq.87 | rep68 | 264 | GCG | |
| seq.88 | rep40 | 264 | GCG | |
| seq.89 | rep78 | 334 | GCG | |
| seq.90 | rep52 | 334 | GCG | |
| seq.91 | rep68 | 334 | GCG | |
| seq.92 | rep40 | 334 | GCG | |
| seq.93 | rep78 | 335 | GCT | |
| seq.94 | rep52 | 335 | GCT | |
| seq.95 | rep68 | 335 | GCT | |
| seq.96 | rep40 | 335 | GCT | |
| seq.97 | rep78 | 337 | GCT | |
| seq.98 | rep52 | 337 | GCT | |
| seq.99 | rep68 | 337 | GCT | |
| seq.100 | rep40 | 337 | GCT | |
| seq.101 | rep78 | 341 | GCC | |
| seq.102 | rep52 | 341 | GCC | |
| seq.103 | rep68 | 341 | GCC | |
| seq.104 | rep40 | 341 | GCC | |
| seq.105 | rep78 | 342 | GCC | |
| seq.106 | rep52 | 342 | GCC | |
| seq.107 | rep68 | 342 | GCC | |
| seq.108 | rep40 | 342 | GCC | |
| seq.109 | rep78 | 347 | GCA | |
| seq.110 | rep52 | 347 | GCA | |
| seq.111 | rep68 | 347 | GCA | |
| seq.112 | rep40 | 347 | GCA | |
| seq.113 | rep78 | 350 | AAT | |
| seq.114 | rep52 | 350 | AAT | |
| seq.115 | rep68 | 350 | AAT | |
| seq.116 | rep40 | 350 | AAT | |
| seq.117 | rep78 | 350 | GCT | |
| seq.118 | rep52 | 350 | GCT | |
| seq.119 | rep68 | 350 | GCT | |
| seq.120 | rep40 | 350 | GCT | |
| seq.121 | rep78 | 354 | GCC | |
| seq.122 | rep52 | 354 | GCC | |
| seq.123 | rep68 | 354 | GCC | |
| seq.124 | rep40 | 354 | GCC | |
| seq.125 | rep78 | 363 | GCC | |
| seq.126 | rep52 | 363 | GCC | |
| seq.127 | rep68 | 363 | GCC | |
| seq.128 | rep40 | 363 | GCC | |
| seq.129 | rep78 | 364 | GCT | |
| seq.130 | rep52 | 364 | GCT | |
| seq.131 | rep68 | 364 | GCT | |
| seq.132 | rep40 | 364 | GCT | |
| seq.133 | rep78 | 367 | GCC | |
| seq.134 | rep52 | 367 | GCC | |
| seq.135 | rep68 | 367 | GCC | |
| seq.136 | rep40 | 367 | GCC | |
| seq.137 | rep78 | 370 | GCC | |
| seq.138 | rep52 | 370 | GCC | |
| seq.139 | rep68 | 370 | GCC | |
| seq.140 | rep40 | 370 | GCC | |
| seq.141 | rep78 | 376 | GCG | |
| seq.142 | rep52 | 376 | GCG | |
| seq.143 | rep68 | 376 | GCG | |
| seq.144 | rep40 | 376 | GCG | |
| seq.145 | rep78 | 381 | GCG | |
| seq.146 | rep52 | 381 | GCG | |
| seq.147 | rep68 | 381 | GCG | |
| seq.148 | rep40 | 381 | GCG | |
| seq.149 | rep78 | 382 | GCG | |
| seq.150 | rep52 | 382 | GCG | |
| seq.151 | rep68 | 382 | GCG | |
| seq.152 | rep40 | 382 | GCG | |
| seq.153 | rep78 | 389 | GCG | |
| seq.154 | rep52 | 389 | GCG | |
| seq.155 | rep68 | 389 | GCG | |
| seq.156 | rep40 | 389 | GCG | |
| seq.157 | rep78 | 407 | GCG | |
| seq.158 | rep52 | 407 | GCC | |
| seq.159 | rep68 | 407 | GCC | |
| seq.160 | rep40 | 407 | GCC | |
| seq.161 | rep78 | 411 | GCA | |
| seq.162 | rep52 | 411 | GCA | |
| seq.163 | rep68 | 411 | GCA | |
| seq.164 | rep40 | 411 | GCA | |
| seq.165 | rep78 | 414 | GCT | |
| seq.166 | rep52 | 414 | GCT | |
| seq.167 | rep68 | 414 | GCT | |
| seq.168 | rep40 | 414 | GCT | |
| seq.169 | rep78 | 420 | GCT | |
| seq.170 | rep52 | 420 | GCT | |
| seq.171 | rep68 | 420 | GCT | |
| seq.172 | rep40 | 420 | GCT | |
| seqA73 | | rep78 | 421 | GCC |
| seq.174 | rep52 | 421 | GCC | |
| seq.175 | rep68 | 421 | GCC | |
| seq.176 | rep40 | 421 | GCC | |
| seq.177 | rep78 | 422 | GCC | |
| seq.178 | rep52 | 422 | GCC | |
| seq.179 | rep68 | 422 | GCC | |
| seq.180 | rep40 | 422 | GCC | |
| seq.181 | rep78 | 424 | GCG | |
| seq.182 | rep52 | 424 | GCG | |
| seq.183 | rep68 | 424 | GCG | |
| seq.184 | rep40 | 424 | GCG | |
| seq.185 | rep78 | 428 | GCT | |
| seq.186 | rep52 | 428 | GCT | |
| seq.187 | rep68 | 428 | GCT | |
| seq.188 | rep40 | 428 | GCT | |
| seq.189 | rep78 | 429 | GCC | |
| seq.190 | rep52 | 429 | GCC | |
| seq.191 | rep68 | 429 | GCC | |
| seq.192 | rep40 | 429 | GCC | |
| seq.193 | rep78 | 438 | GCG | |
| seq.194 | rep52 | 438 | GCG | |
| seq.195 | rep68 | 438 | GCG | |
| seq.196 | rep40 | 438 | GCG | |
| seq.197 | rep78 | 440 | GCG | |
| seq.198 | rep52 | 440 | GCG | |
| seq.199 | rep68 | 440 | GCG | |
| seq.200 | rep40 | 440 | GCG | |
| seq.201 | rep78 | 451 | GCC | |
| seq.202 | rep52 | 451 | GCC | |
| seq.203 | rep68 | 451 | GCC | |
| seq.204 | rep40 | 451 | GCC | |
| seq.205 | rep78 | 460 | GCG | |
| seq.206 | rep52 | 460 | GCG | |
| seq.207 | rep68 | 460 | GCG | |
| seq.208 | rep40 | 460 | GCG | |
| seq.209 | rep78 | 462 | GCC | |
| seq.210 | rep52 | 462 | GCC | |
| seq.211 | rep68 | 462 | GCC | |
| seq.212 | rep40 | 462 | GCC | |
| seq.213 | rep78 | 462 | ATA | |
| seq.214 | rep52 | 462 | ATA | |
| seq.215 | rep68 | 462 | ATA | |
| seq.216 | rep40 | 462 | ATA | |
| seq.217 | rep78 | 484 | GCC | |
| seq.218 | rep52 | 484 | GCG | |
| seq.219 | rep68 | 484 | GCC | |
| seq.220 | rep40 | 484 | GCC | |
| seq.221 | rep78 | 488 | GCG | |
| seq.222 | rep52 | 488 | GCG | |
| seq.223 | rep68 | 488 | GCG | |
| seq.224 | rep40 | 488 | GCG | |
| seq.225 | rep78 | 495 | GCC | |
| seq.226 | rep52 | 495 | GCC | |
| seq.227 | rep68 | 495 | GCC | |
| seq.228 | rep40 | 495 | GCC | |
| seq.229 | rep78 | 497 | GCC | |
| seq.230 | rep52 | 497 | GCC | |
| seq.231 | rep68 | 497 | GCC | |
| seq.232 | rep40 | 497 | GCC | |
| seq.233 | rep78 | 497 | CGA | |
| seq.234 | rep52 | 497 | CGA | |
| seq.235 | rep68 | 497 | CGA | |
| seq.236 | rep40 | 497 | CGA | |
| seq.237 | rep78 | 497 | CTC | |
| seq.238 | rep52 | 497 | CTC | |
| seq.239 | rep68 | 497 | CTC | |
| seq.240 | rep40 | 497 | CTC | |
| seq.241 | rep78 | 497 | TAC | |
| seq.242 | rep52 | 497 | TAC | |
| seq.243 | rep68 | 497 | TAC | |
| seq.244 | rep40 | 497 | TAC | |
| seq.245 | rep78 | 498 | GCT | |
| seq.246 | rep52 | 498 | GCT | |
| seq.247 | rep68 | 498 | GCT | |
| seq.248 | rep40 | 498 | GCT | |
| seq.249 | rep78 | 499 | GCC | |
| seq.250 | rep52 | 499 | GCC | |
| seq.251 | rep68 | 499 | GCC | |
| seq.252 | rep40 | 499 | GCC | |
| seq.253 | rep78 | 503 | GCC | |
| seq.254 | rep52 | 503 | GCG | |
| seq.255 | rep68 | 503 | GCG | |
| seq.256 | rep40 | 503 | GCG | |
| seq.257 | rep78 | 510 | GCA | |
| seq.258 | rep52 | 510 | GCA | |
| seq.259 | rep68 | 510 | GCA | |
| seq.260 | rep40 | 510 | GCA | |
| seq.261 | rep78 | 511 | GCA | |
| seq.262 | rep52 | 511 | GCA | |
| seq.263 | rep68 | 511 | GCA | |
| seq.264 | rep40 | 511 | GCA | |
| seq.265 | rep78 | 512 | GCT | |
| seq.266 | rep52 | 512 | GCT | |
| seq.267 | rep68 | 512 | GCT | |
| seq.268 | rep40 | 512 | GCT | |
| seq.269 | rep78 | 516 | GCG | |
| seq.270 | rep52 | 516 | GCG | |
| seq.271 | rep68 | 516 | GCG | |
| seq.272 | rep40 | 516 | GCG | |
| seq.273 | rep78 | 517 | GCT | |
| seq.274 | rep52 | 517 | GCT | |
| seq.275 | rep68 | 517 | GCT | |
| seq.276 | rep40 | 517 | GCT | |
| seq.277 | rep78 | 517 | AAC | |
| seq.278 | rep52 | 517 | AAC | |
| seq.279 | rep68 | 517 | AAC | |
| seq.280 | rep40 | 517 | AAC | |
| seq.281 | rep78 | 518 | GCA | |
| seq.282 | rep52 | 518 | GCA | |
| seq.283 | rep68 | 518 | GCA | |
| seq.284 | rep40 | 518 | GCA | |
| seq.285 | rep78 | 519 | GCG | |
| seq.286 | rep52 | 519 | GCG | |
| seq.287 | rep68 | 519 | GCG | |
| seq.288 | rep40 | 519 | GCG | |
| seq.289 | rep78 | 598 | GCA | |
| seq.290 | rep52 | 598 | GCA | |
| seq.291 | rep78 | 598 | GAC | |
| seq.292 | rep52 | 598 | GAC | |
| seq.293 | rep78 | 598 | AGC | |
| seq.294 | rep52 | 598 | AGC | |
| seq.295 | rep78 | 600 | GCG | |
| seq.296 | rep52 | 600 | GCG | |
| seq.297 | rep78 | 600 | CCG | |
| seq.298 | rep52 | 600 | CCG | |
| seq.299 | rep78 | 601 | GCA | |
| seq.300 | rep52 | 601 | GCA | |
| seq.301 | rep78 | 335 420 495 | GCT GCC GCC | |
| seq.302 | rep52 | 335 420 495 | GCT GCC GCC | |
| seq.303 | rep68 | 335 420 495 | GCT GCC GCC | |
| seq.304 | rep40 | 335 420 495 | GCT GCC GCC | |
| seq.305 | rep78 | 39 140 | GCA GCC | |
| seq.306 | rep68 | 39 140 | GCA GCC | |
| seq.307 | rep78 | 279 428 451 | GCC GCT GCC | |
| seq.308 | rep52 | 279 428 451 | GCC GCT GCC | |
| seq.309 | rep68 | 279 428 451 | GCC GCT GCC | |
| seq.310 | rep40 | 279 428 451 | GCC GCT GCC | |
| seq.311 | rep78 | 125 237 600 | GCG GCC GCG | |
| seq.312 | rep52 | 125 237 600 | GCG GCC GCG | |
| seq.313 | rep68 | 125 237 600 | GCG GCC GCG | |
| seq.314 | rep40 | 125 237 600 | GCG GCC GCG | |
| seq.315 | rep78 | 163 259 | GCT GCG | |
| seq.316 | rep52 | 163 259 | GCT GCG | |
| seq.317 | rep68 | 163 259 | GCT GCG | |
| seq.318 | rep40 | 163 259 | GCT GCG | |
| seq.319 | rep78 | 17 127 189 | GCG GCT GCG | |
| seq.320 | rep68 | 17 127 189 | GCG GCT GCG | |
| seq.321 | rep78 | 350 428 | GCT GCT | |
| seq.322 | rep52 | 350 428 | GCT GCT | |
| seq.323 | rep68 | 350 428 | GCT GCT | |
| seq.324 | rep40 | 350 428 | GCT GCT | |
| seq.325 | rep78 | 54 338 495 | GCC GCC GCC | |
| seq.326 | rep52 | 54 338 495 | GCC GCC GCC | |
| seq.327 | rep68 | 54 338 495 | GCC GCC GCC | |
| seq.328 | rep40 | 54 338 495 | GCC GCC GCC | |
| seq.329 | rep78 | 350 420 | GCT GCC | |
| seq.330 | rep52 | 350 420 | GCT GCC | |
| seq.331 | rep68 | 350 420 | GCT GCC | |
| seq.332 | rep40 | 350 420 | GCT GCC | |
| seq.333 | rep78 | 189 197 518 | GCG GCG GCA | |
| seq.334 | rep52 | 189 197 518 | GCG GCG GCA | |
| seq.335 | rep68 | 189 197 518 | GCG GCG GCA | |
| seq.336 | rep40 | 189 197 518 | GCG GCG GCA | |
| seq.337 | rep78 | 468 516 | GCC GCG | |
| seq.338 | rep52 | 468 516 | GCC GCG | |
| seq.339 | rep68 | 468 516 | GCC GCG | |
| seq.340 | rep40 | 468 516 | GCC GCG | |
| seq.341 | rep78 | 127 221 350 54 | GCT GCA GCT GCC GCC | |
| | | 140 | | |
| seq.342 | rep52 | 127 221 350 54 | GCT GCA GCT GCC GCC | |
| | | 140 | | |
| seq.343 | rep68 | 127 221 350 54 | GCT GCA GCT GCC GCC | |
| | | 140 | | |
| seq.344 | rep40 | 127 221 350 54 | GCT GCA GCT GCC GCC | |
| | | 140 | | |
| seq.345 | rep78 | 221 285 | GCA GCG | |
| seq.346 | rep52 | 221 285 | GCA GCG | |
| seq.347 | rep68 | 221 285 | GCA GCG | |
| seq.348 | rep40 | 221 285 | GCA GCG | |
| seq.349 | rep78 | 23 495 | GCT GCC | |
| seq.350 | rep52 | 23 495 | GCT GCC | |
| seq.351 | rep68 | 23 495 | GCT GCC | |
| seq.352 | rep40 | 23 495 | GCT GCC | |
| seq.353 | rep78 | 20 54 420 495 | GCC GCC GCC GCC | |
| seq.354 | rep52 | 20 54 420 495 | GCC GCC GCC GCC | |
| seq.355 | rep68 | 20 54 420 495 | GCC GCC GCC GCC | |
| seq.356 | rep40 | 20 54 420 495 | GCC GCC GCC GCC | |
| seq.357 | rep78 | 412 612 | GCC GCG | |
| seq.358 | rep52 | 412 612 | GCC GCG | |
| seq.359 | rep68 | 412 612 | GCC GCG | |
| seq.360 | rep40 | 412 612 | GCC GCG | |
| seq.361 | rep78 | 197 412 | GCG GCC | |
| seq.362 | rep52 | 197 412 | GCG GCC | |
| seq.363 | rep68 | 197 412 | GCG GCC | |
| seq.364 | rep40 | 197 412 | GCG GCC | |
| seq.365 | rep78 | 412 495 511 | GCC GCC GGA | |
| seq.366 | rep52 | 412 495 511 | GCC GCC GCA | |
| seq.367 | rep68 | 412 495 511 | GCC GCC GCA | |
| seq.368 | rep40 | 412 495 511 | GCC GCC GCA | |
| seq.369 | rep78 | 98 422 | GCC GCC | |
| seq.370 | rep52 | 98 422 | GCC GCC | |
| seq.371 | rep68 | 98 422 | GCC GCC | |
| seq.372 | rep40 | 98 422 | GCC GCC | |
| seq.373 | rep78 | 17 127 189 | GCG GCT GCG | |
| seq.374 | rep68 | 17 127 189 | GCG GCT GCG | |
| seq.375 | rep78 | 20 54 495 | GCC GCC GCC | |
| seq.376 | rep52 | 20 54 495 | GCC GCC GCC | |
| seq.377 | rep68 | 20 54 495 | GCC GCC GCC | |
| seq.378 | rep40 | 20 54 495 | GCC GCC GCC | |
| seq.379 | rep78 | 259 54 | GCG GCC | |
| seq.380 | rep52 | 259 54 | GCG GCC | |
| seq.381 | rep68 | 259 54 | GCG GCC | |
| seq.382 | rep40 | 259 54 | GCG GCC | |
| seq.383 | rep78 | 335 399 | GCT GCG | |
| seq.384 | rep52 | 335 399 | GCT GCG | |
| seq.385 | rep68 | 335 399 | GCT GCG | |
| seq.386 | rep40 | 335 399 | GCT GCG | |
| seq.387 | rep78 | 221 432 | GCA GCA | |
| seq.388 | rep52 | 221 432 | GCA GCA | |
| seq.389 | rep68 | 221 432 | GCA GCA | |
| seq.390 | rep40 | 221 432 | GCA GCA | |
| seq.391 | rep78 | 259 516 | GCG GCG | |
| seq.392 | rep52 | 259 516 | GCG GCG | |
| seq.393 | rep68 | 259 516 | GCG GCG | |
| seq.394 | rep40 | 259 516 | GCG GCG | |
| seq.395 | rep78 | 495 516 | GCC GCG | |
| seq.396 | rep52 | 495 516 | GCC GCG | |
| seq.397 | rep68 | 495 516 | GCC GCG | |
| seq.398 | rep40 | 495 516 | GCC GCG | |
| seq.399 | rep78 | 414 14 | GCT GCC | |
| seq.400 | rep52 | 414 14 | GCT GCC | |
| seq.401 | rep68 | 414 14 | GCT GCC | |
| seq.402 | rep40 | 414 14 | GCT GCC | |
| seq.403 | rep78 | 74 402 495 | GCG GCC GCC | |
| seq.404 | rep52 | 74 402 495 | GCG GCC GCC | |
| seq.405 | rep68 | 74 402 495 | GCG GCC GCC | |
| seq.406 | rep40 | 74 402 495 | GCG GCC GCC | |
| seq.407 | rep78 | 228 462 497 | GCC GCC GCC | |
| seq.408 | rep52 | 228 462 497 | GCC GCC GCC | |
| seq.409 | rep68 | 228 462 497 | GCC GCC GCC | |
| seq.410 | rep40 | 228 462 497 | GCC GCC GCC | |
| seq.411 | rep78 | 290 338 | GCG GCC | |
| seq.412 | rep52 | 290 338 | GCG GCC | |
| seq.413 | rep68 | 290 338 | GCG GCC | |
| seq.414 | rep40 | 290 338 | GCG GCC | |
| seq.415 | rep78 | 140 511 | GCC GCA | |
| seq.416 | rep52 | 140 511 | GCC GCA | |
| seq.417 | rep68 | 140 511 | GCC GCA | |
| seq.418 | rep40 | 140 511 | GCC GCA | |
| seq.419 | rep78 | 86 378 | GCG GCG | |
| seq.420 | rep52 | 86 378 | GCG GCG | |
| seq.421 | rep68 | 86 378 | GCG GCG | |
| seq.422 | rep40 | 86 378 | GCG GCG | |
| seq.423 | rep78 | 54 86 | GCC GCG | |
| seq.424 | rep68 | 54 86 | GCC GCG | |
| seq.425 | rep78 | 54 86 | GCC GCG | |
| seq.426 | rep68 | 54 86 | GCC GCG | |
| seq.427 | rep78 | 214 495 140 | GCG GCC GCC | |
| seq.428 | rep52 | 214 495 140 | GCG GCC GCC | |
| seq.429 | rep68 | 214 495 140 | GCG GCC GCC | |
| seq.430 | rep40 | 214 495 140 | GCG GCC GCC | |
| seq.431 | rep78 | 495 511 | GCC GCA | |
| seq.432 | rep52 | 495 511 | GCC GCA | |
| seq.433 | rep68 | 495 511 | GCC GCA | |
| seq.434 | rep40 | 495 511 | GCC GCA | |
| seq.435 | rep78 | 495 54 | GCC GCC | |
| seq.436 | rep52 | 495 54 | GCC GGC | |
| seq.437 | rep68 | 495 54 | GCC GCC | |
| seq.438 | rep40 | 495 54 | GCC GCC | |
| seq.439 | rep78 | 197 495 | GCG GCC | |
| seq.440 | rep52 | 197 495 | GCG GCC | |
| seq.441 | rep68 | 197 495 | GCG GCG | |
| seq.442 | rep40 | 197 495 | GCG GCC | |
| seq.443 | rep78 | 261 20 | GCC GCC | |
| seq.444 | rep52 | 261 20 | GCG GCC | |
| seq.445 | rep68 | 261 20 | GCC GCC | |
| seq.446 | rep40 | 261 20 | GCC GCC | |
| seq.447 | rep78 | 54 20 | GCC GCC | |
| seq.448 | rep68 | 54 20 | GCC GCC | |
| seq.449 | rep78 | 197 420 | GCG GCC | |
| seq.450 | rep52 | 197 420 | GCG GCC | |
| seq.451 | rep68 | 197 420 | GCG GCC | |
| seq.452 | rep40 | 197 420 | GCG GCC | |
| seq.453 | rep78 | 54 338 495 | GCC GCC GCC | |
| seq.454 | rep52 | 54 338 495 | GCC GCC GCC | |
| seq.455 | rep68 | 54 338 495 | GCC GCC GCC | |
| seq.456 | rep40 | 54 338 495 | GCC GCC GCC | |
| seq.457 | rep78 | 197 427 | GCG GCG | |
| seq.458 | rep52 | 197 427 | GCG GCG | |
| seq.459 | rep68 | 197 427 | GCG GCG | |
| seq.460 | rep40 | 197 427 | GCG GCG | |
| seq.461 | rep78 | 54 228 370 387 | GCC GCC GCC GCG | |
| seq.462 | rep52 | 54 228 370 387 | GCC GCC GCC GCG | |
| seq.463 | rep68 | 54 228 370 387 | GCC GCC GCC GCG | |
| seq.464 | rep40 | 54 228 370 387 | GCC GCC GCG GCG | |
| seq.465 | rep78 | 221 289 | GCA GCC | |
| seq.466 | rep52 | 221 289 | GCA GCG | |
| seq.467 | rep68 | 221 289 | GCA GCC | |
| seq.468 | rep40 | 221 289 | GGA GCG | |
| seq.469 | rep78 | 54 163 | GCC GCT | |
| seq.470 | rep68 | 54 163 | GCC GCT | |
| seq.471 | rep78 | 341 407 420 | GCC GCC GCC | |
| seq.472 | rep52 | 341 407 420 | GCC GCC GCC | |
| seq.473 | rep68 | 341 407 420 | GCC GCC GCC | |
| seq.474 | rep40 | 341 407 420 | GCC GCC GCC | |
| seq.475 | rep78 | 54 228 | GCC GCC | |
| seq.476 | rep52 | 54 228 | GGC GCC | |
| seq.477 | rep68 | 54 228 | GCC GCC | |
| seq.478 | rep40 | 54 228 | GCC GCC | |
| seq.479 | rep78 | 96 125 511 | GCA GCG GCA | |
| seq.480 | rep52 | 96 125 511 | GCA GCG GCA | |
| seq.481 | rep68 | 96 125 511 | GCA GCG GCA | |
| seq.482 | rep40 | 96 125 511 | GCA GGG GCA | |
| seq.483 | rep78 | 54 163 | GCC GCT | |
| seq.484 | rep68 | 54 163 | GCC GCT | |
| seq.485 | rep78 | 197 420 | GCG GCC | |
| seq.486 | rep52 | 197 420 | GCG GCC | |
| seq.487 | rep68 | 197 420 | GCG GCC | |
| seq.488 | rep40 | 197 420 | GCG GCC | |
| seq.489 | rep78 | 334 428 499 | GCG GCT GCC | |
| seq.490 | rep52 | 334 428 499 | GCG GCT GCC | |
| seq.491 | rep68 | 334 428 499 | GCG GCT GCG | |
| seq.492 | rep40 | 334 428 499 | GCG GCT GCC | |
| seq.493 | rep78 | 197 414 | GCG GCT | |
| seq.494 | rep52 | 197 414 | GCG GCT | |
| seq.495 | rep68 | 197 414 | GCG GCT | |
| seq.496 | rep40 | 197 414 | GCG GCT | |
| seq.497 | rep78 | 30 54 127 | GCG GCC GCT | |
| seq.498 | rep68 | 30 54 127 | GCG GCC GCT | |
| seq.499 | rep78 | 29 260 | GCG GCG | |
| seq.500 | rep52 | 29 260 | GCG GCG | |
| seq.501 | rep68 | 29 260 | GCG GCG | |
| seq.502 | rep40 | 29 260 | GCG GCG | |
| seq.503 | rep78 | 4 484 | GCT GCC | |
| seq.504 | rep52 | 4 484 | GCT GCC | |
| seq.505 | rep68 | 4 484 | GCT GCC | |
| seq.506 | rep40 | 4 484 | GCT GCC | |
| seq.507 | rep78 | 258 124 132 | GCC GCC GCC | |
| seq.508 | rep52 | 258 124 132 | GCC GCC GCC | |
| seq.509 | rep68 | 258 124 132 | GCC GCC GCC | |
| seq.510 | rep40 | 258 124 132 | GCC GCC GCC | |
| seq.511 | rep78 | 231 497 | GCC GCC | |
| seq.512 | rep52 | 231 497 | GCC GCC | |
| seq.513 | rep68 | 231 497 | GCC GCC | |
| seq.514 | rep40 | 231 497 | GCC GCC | |
| seq.515 | rep78 | 221 258 | GCA GCC | |
| seq.516 | rep52 | 221 258 | GCA GCC | |
| seq.517 | rep68 | 221 258 | GCA GCC | |
| seq.518 | rep40 | 221 258 | GCA GCC | |
| seq.519 | rep78 | 234 264 326 | GCG GCG GCC | |
| seq.520 | rep52 | 234 264 326 | GCG GCG GCC | |
| seq.521 | rep68 | 234 264 326 | GCG GCG GCC | |
| seq.522 | rep40 | 234 264 326 | GCG GCG GCC | |
| seq.523 | rep78 | 153 398 | AGC GCG | |
| seq.524 | rep52 | 153 398 | AGC GCG | |
| seq.525 | rep68 | 153 398 | AGC GCG | |
| seq.526 | rep40 | 153 398 | AGC GCG | |
| seq.527 | rep78 | 53 216 | GCG GCC | |
| seq.528 | rep68 | 53 216 | GCG GCC | |
| seq.529 | rep78 | 22 382 | GCT GCG | |
| seq.530 | rep52 | 22 382 | GCT GCG | |
| seq.531 | rep68 | 22 382 | GCT GCG | |
| seq.532 | rep40 | 22 382 | GCT GCG | |
| seq.533 | rep78 | 231 411 | GGC GCA | |
| seq.534 | rep52 | 231 411 | GGC GCA | |
| seq.535 | rep68 | 231 411 | GCG GCA | |
| seq.536 | rep40 | 231 411 | GCC GCA | |
| seq.537 | rep78 | 59 305 | GCG GCC | |
| seq.538 | rep52 | 59 305 | GCG GCC | |
| seq.539 | rep68 | 59 305 | GCG GCC | |
| seq.540 | rep40 | 59 305 | GCG GCC | |
| seq.541 | rep78 | 53 231 | GCG GCC | |
| seq.542 | rep52 | 53 231 | GCG GCC | |
| seq.543 | rep68 | 53 231 | GCG GCC | |
| seq.544 | rep40 | 53 231 | GCG GGC | |
| seq.545 | rep78 | 258 498 | GCC GCT | |
| seq.546 | rep52 | 258 498 | GCC GCT | |
| seq.547 | rep68 | 258 498 | GCC GCT | |
| seq.548 | rep40 | 258 498 | GCC GCT | |
| seq.549 | rep78 | 88 231 | GCC GCC | |
| seq.550 | rep52 | 88 231 | GCC GCC | |
| seq.551 | rep68 | 88 231 | GCC GCC | |
| seq.552 | rep40 | 88 231 | GCC GCC | |
| seq.553 | rep78 | 101 363 | GCA GCC | |
| seq.554 | rep52 | 101 363 | GCA GCC | |
| seq.555 | rep68 | 101 363 | GCA GGC | |
| seq.556 | rep40 | 101 363 | GCA GCC | |
| seq.557 | rep78 | 354 132 | GCC GCC | |
| seq.558 | rep52 | 354 132 | GCC GCC | |
| seq.559 | rep68 | 354 132 | GCC GGC | |
| seq.560 | rep40 | 354 132 | GCC GCC | |
| seq.561 | rep78 | 10 132 | GCG GCC | |
| seq.562 | rep68 | 10 132 | GCG GCC | |

DNA Sequences

| Sequence | aa position | codon |
|---|---|---|
| seq.563 | 4 | GCT |
| seq.564 | 10 | GCG |
| seq.565 | 20 | GCC |
| seq.566 | 22 | GCT |
| seq.567 | 29 | GCG |
| seq.568 | 38 | GCG |
| seq.569 | 39 | GCA |
| seq.570 | 53 | GCT |
| seq.571 | 59 | GCG |
| seq.572 | 64 | GCT |
| seq.573 | 74 | GCG |
| seq.574 | 86 | GCG |
| seq.575 | 88 | GCC |
| seq.576 | 101 | GCA |
| seq.577 | 124 | GCC |
| seq.578 | 125 | GCG |
| seq.579 | 127 | GCT |
| seq.580 | 132 | GCC |
| seq.581 | 140 | GCC |
| seq.582 | 161 | GCC |
| seq.583 | 163 | GCT |
| seq.584 | 175 | GCT |
| seq.585 | 193 | GCG |
| seq.586 | 196 | GCC |
| seq.587 | 197 | GCC |
| seq.588 | 221 | GCA |
| seq.589 | 228 (Rep78/68) | GCG |
| | 228 (Rep52) | GCG |
| | 228 (Rep 40) | GCG |
| seq.590 | 231 (Rep78/68) | GCC |
| | 231 (Rep 52) | GCC |
| | 231 (Rep 40) | GCC |
| seq.591 | 234 (Rep78/68) | GCG |
| | 234 (Rep 52) | GCG |
| | 234 (Rep 40) | GCG |
| seq.592 | 237 (Rep78/68) | GCC |
| | 237 (Rep 52) | GCC |
| | 237 (Rep 40) | GCC |
| seq.593 | 250 (Rep78/68) | GCC |
| | 250 | GCC |
| | 250 | GCC |
| seq.594 | 258 (Rep78/68) | GCC |
| | 258 | GCC |
| | 258 | GCC |
| seq.595 | 260 (Rep78/68) | GCG |
| | 260 | GCG |
| | 260 | GCG |
| seq.596 | 263 (Rep78/68) | GCC |
| | 263 | GCC |
| | 263 | GCC |
| seq.597 | 264 (Rep78/68) | GCG |
| | 264 | GCG |
| | 264 | GCG |
| seq.598 | 334 (Rep78/68) | GCG |
| | 334 | GCG |
| | 334 | GCG |
| seq.599 | 335 (Rep78/68) | GCT |
| | 335 | GCT |
| | 335 | GCT |
| seq.600 | 337 (Rep78/68) | GCT |
| | 337 | GCT |
| | 337 | GCT |
| seq.601 | 341 (Rep78/68) | GCC |
| | 341 | GCC |
| | 341 | GCC |
| seq.602 | 342 (Rep78/68) | GCC |
| | 342 | GCC |
| | 342 | GCC |
| seq.603 | 347 (Rep78/68) | GCA |
| | 347 | GCA |
| | 347 | GCA |
| seq.604 | 350 (Rep78/68) | AAT |
| | 350 | AAT |
| | 350 | AAT |
| seq.605 | 350 (Rep78/68) | GCT |
| | 350 | GCT |
| | 350 | GCT |
| seq.606 | 354 (Rep78/68) | GCC |
| | 354 | GCC |
| | 354 | GCC |
| seq.607 | 363 (Rep78/68) | GCC |
| | 363 | GCC |
| | 363 | GCC |
| seq.608 | 364 (Rep78/68) | GCT |
| | 364 | GCT |
| | 364 | GCT |
| seq.609 | 367 (Rep78/68) | GCC |
| | 367 | GCC |
| | 367 | GCC |
| seq.610 | 370 (Rep78/68) | GCC |
| | 370 | GCC |
| | 370 | GCC |
| seq.611 | 376 (Rep78/68) | GCG |
| | 376 | GCG |
| | 376 | GCG |
| seq.612 | 381 (Rep78/68) | GCG |
| | 381 | GCG |
| | 381 | GCG |
| seq.613 | 382 (Rep78/68) | GCG |
| | 382 | GCG |
| | 382 | GCG |
| seq.614 | 389 (Rep78/68) | GCG |
| | 389 | GCG |
| | 389 | GCG |
| seq.615 | 407 (Rep78/68) | GCC |
| | 407 | GCC |
| | 407 | GCC |
| seq.616 | 411 (Rep78/68) | GCA |
| | 411 | GCA |
| | 411 | GCA |
| seq.617 | 414 (Rep78/68) | GCT |
| | 414 | GCT |
| | 414 | GCT |
| seq.618 | 420 (Rep78/68) | GCT |
| | 420 | GCT |
| | 420 | GCT |
| seq.619 | 421 (Rep78/68) | GCC |
| | 421 | GCC |
| | 421 | GCC |
| seq.620 | 422 (Rep78/68) | GCC |
| | 422 | GCC |
| | 422 | GCC |
| seq.621 | 424 (Rep78/68) | GCG |
| | 424 | GCG |
| | 424 | GCG |
| seq.622 | 428 (Rep78/68) | GCT |
| | 428 | GCT |
| | 428 | GCT |
| seq.623 | 429 (Rep78/68) | GCC |
| | 429 | GCC |
| | 429 | GCC |
| seq.624 | 438 (Rep78/68) | GCG |
| | 438 | GCG |
| | 438 | GCG |
| seq.625 | 440 (Rep78/68) | GCG |
| | 440 | GCG |
| | 440 | GCG |
| seq.626 | 451 (Rep78/68) | GCC |
| | 451 | GCC |
| | 451 | GCC |
| seq.627 | 460 (Rep78/68) | GCG |
| | 460 | GCG |
| | 460 | GCG |
| seq.628 | 462 (Rep78/68) | GCC |
| | 462 | GCC |
| | 462 | GCC |
| seq.629 | 462 (Rep78/68) | ATA |
| | 462 | ATA |
| | 462 | ATA |
| seq.630 | 484 (Rep78/68) | GCC |
| | 484 | GCC |
| | 484 | GCC |
| seq.631 | 488 (Rep78/68) | GCG |
| | 488 | GCG |
| | 488 | GCG |
| seq.632 | 495 (Rep78/68) | GCC |
| | 495 | GCC |
| | 495 | GCC |
| seq.633 | 497 (Rep78/68) | GCC |
| | 497 | GCC |
| | 497 | GCC |
| seq.634 | 497 (Rep78/68) | CGA |
| | 497 | CGA |
| | 497 | CGA |
| seq.635 | 497 (Rep78/68) | CTC |
| | 497 | CTC |
| | 497 | CTC |
| seq.636 | 497 (Rep78/68) | TAC |
| | 497 | TAC |
| | 497 | TAC |
| seq.637 | 498 (Rep78/68) | GCT |
| | 498 | GCT |
| | 498 | GCT |
| seq.638 | 499 (Rep78/68) | GCC |
| | 499 | GCC |
| | 499 | GCC |
| seq.639 | 503 (Rep78/68) | GCG |
| | 503 | GCG |
| | 503 | GCG |
| seq.640 | 510 (Rep78/68) | GCA |
| | 510 | GCA |
| | 510 | GCA |
| seq.641 | 511 (Rep78/68) | GCA |
| | 511 | GCA |
| | 511 | GCA |
| seq.642 | 512 (Rep78/68) | GCT |
| | 512 | GCT |
| | 512 | GCT |
| seq.643 | 516 (Rep78/68) | GCG |
| | 516 | GCG |
| | 516 | GCG |
| seq.644 | 517 (Rep78/68) | GCT |
| | 517 | GCT |
| | 517 | GCT |
| seq.645 | 517 (Rep78/68) | AAC |
| | 517 | AAC |
| | 517 | AAC |
| seq.646 | 518 (Rep78/68) | GCA |
| | 518 | GCA |
| | 518 | GCA |
| seq.647 | 519 (Rep78/68) | GCG |
| | 519 | GCG |
| | 519 | GCG |
| seq.648 | 598 (Rep78/68) | GCA |
| seq.649 | 600 (Rep78/68) | GCG |
| seq.650 | 601 (Rep78/68) | GCA |
| seq.651 | 335 420 495 | GCT GCC GCC |
| | 335 420 495 | GCT GCC GCC |
| | 335 420 495 | GCT GGC GCC |
| seq.652 | 39 140 | GCA GCC |
| seq.653 | 279 428 451 | GCC GCT GCC |
| | 279 428 451 | GCC GCT GCC |
| | 279 428 451 | GCC GCT GCC |
| seq.654 | 125 237 600 | GCG GCC GCG |
| | 125 237 600 | GCG GCC GCG |
| | 125 237 600 | GCG GCC GCG |
| seq.655 | 163 259 | GCT GCG |
| | 163 259 | GCT GCG |
| | 163 259 | GCT GC G |
| seq.656 | 17 127 189 | GCG GCT GCG |
| seq.657 | 350 428 | GCT GCT |
| | 350 428 | GCT GCT |
| | 350 428 | GCT GCT |
| seq.658 | 54 338 495 | GCC GCC GCC |
| | 54 338 495 | GCC GCC GCC |
| | 54 338 495 | GCC GCC GCC |
| seq.659 | 350 420 | GCT GCC |
| | 350 420 | GCT GCC |
| | 350 420 | GCT GCC |
| seq.660 | 189 197 518 | GCG GCG GCA |
| | 189 197 518 | GCG GCG GCA |
| | 189 197 518 | GCG GCG GCA |
| seq.661 | 468 516 | GCC GCG |
| | 468 516 | GCC GCG |
| | 468 516 | GCC GCG |
| seq.662 | 127 221 350 54 140 | GCT GCA GCT GCC GCC |
| | 127 221 350 54 140 | GCT GCA GCT GCC GCC |
| | 127 221 350 54 140 | GCT GCA GCT GCC GCC |
| seq.663 | 221 285 | GCA GCG |
| | 221 285 | GCA GCG |
| | 221 285 | GCA GCG |
| seq.664 | 23 495 | GCT GCC |
| | 23 495 | GCT GCC |
| | 23 495 | GCT GCC |
| seq.665 | 20 54 420 495 | GCC GCC GCC GCC |
| | 20 54 420 495 | GCC GCC GCC GCC |
| | 20 54 420 495 | GCC GCC GCC GCC |
| seq.666 | 412 612 | GCC GCG |
| | 412 612 | GCC GCG |
| | 412 612 | GCC GCG |
| seq.667 | 197 412 | GCG GCC |
| | 197 412 | GCG GCC |
| | 197 412 | GCG GCC |
| seq.668 | 412 495 511 | GCC GCC GCA |
| | 412 495 511 | GCC GCC GCA |
| | 412 495 511 | GCC GCC GCA |
| seq.669 | 98 422 | GCC GCC |
| | 98 422 | GCC GCC |
| | 98 422 | GCC GCC |
| seq.670 | 17 127 189 | GCG GCT GCG |
| seq.671 | 20 54 495 | GCC GCC GCC |
| | 20 54 495 | GCC GCC GCC |
| | 20 54 495 | GCC GCC GCC |
| seq.672 | 54 163 | GCC GCT |
| seq.673 | 259 54 | GCG GCC |
| | 259 54 | GCG GCC |
| | 259 54 | GCG GCC |
| seq.674 | 335 399 | GCT GCG |
| | 335 399 | GCT GCG |
| | 335 399 | GCT GCG |
| seq.675 | 221 432 | GCA GCA |
| | 221 432 | GCA GCA |
| | 221 432 | GCA GCA |
| seq.676 | 259 516 | GCG GCG |
| | 259 516 | GCG GCG |
| | 259 516 | GCG GCG |
| seq.677 | 495 516 | GCC GCG |
| | 495 516 | GCC GCG |
| | 495 516 | GCC GCG |
| seq.678 | 414 14 | GCT GCC |
| | 414 14 | GCT GCC |
| | 414 14 | GCT GCC |
| seq.679 | 74 402 495 | GCG GCC GCC |
| | 74 402 495 | GCG GCC GCC |
| | 74 402 495 | GCG GCC GCC |
| seq.680 | 228 462 497 | GCC GCC GCC |
| | 228 462 497 | GCC GCC GCC |
| | 228 462 497 | GCC GCC GCC |
| seq.681 | 290 338 | GCG GCC |
| | 290 338 | GCG GCC |
| | 290 338 | GCG GCC |
| seq.682 | 140 511 | GCC GCA |
| | 140 511 | GCC GCA |
| | 140 511 | GCC GCA |
| seq.683 | 86 378 | GCG GCG |
| | 86 378 | GCG GCG |
| | 86 378 | GCG GCG |
| seq.684 | 54 86 | GCC GCG |
| | 54 86 | GCC GCG |
| | 54 86 | GCC GCG |
| seq.685 | 214 495 140 | GCG GCC GCC |
| | 214 495 140 | GCG GCC GCC |
| | 214 495 140 | GCG GCC GCC |
| seq.686 | 495 511 | GCC GCA |
| | 495 511 | GCC GCA |
| | 495 511 | GCC GCA |
| seq.687 | 495 54 | GCC GCC |
| | 495 54 | GCC GCC |
| | 495 54 | GCC GCC |
| seq.688 | 197 495 | GCG GCC |
| | 197 495 | GCG GCC |
| | 197 495 | GCG GCC |
| seq.689 | 261 20 | GCC GCC |
| | 261 20 | GCC GCC |
| | 261 20 | GCC GCC |
| seq.690 | 54 20 | GCC GCC |
| seq.691 | 197 420 | GCG GCC |
| | 197 420 | GCG GCC |
| | 197 420 | GCG GCC |
| seq.692 | 54 338 495 | GCC GCC GCC |
| | 54 338 495 | GCC GCC GCC |
| | 54 338 495 | GCC GCC GCC |
| seq.693 | 197 427 | GCG GCG |
| | 197 427 | GCG GCG |
| | 197 427 | GCG GCG |
| seq.694 | 54 228 370 387 | GCC GCC GCC GCG |
| | 54 228 370 387 | GCC GCC GCC GCG |
| | 54 228 370 387 | GCC GCC GCC GCG |
| seq.695 | 221 289 | GCA GCC |
| | 221 289 | GCA GCG |
| | 221 289 | GCAGCC |
| seq.696 | 54 163 | GCC GCT |
| 0 54 163 | GCC GCT | |
| seq.697 | 341 407 420 | GCC GCC GCC |
| | 341 407 420 | GCC GCC GCC |
| | 341 407 420 | GCC GCC GCC |
| seq.698 | 54 228 | GCC GCC |
| | 54 228 | GCC GCC |
| | 54 228 | GCC GCC |
| seq.699 | 96 125 511 | GCA GCG GCA |
| | 96 125 511 | GCA GCG GCA |
| | 96 125 511 | GCA GCG GCA |
| seq.700 | 197 420 | GCG GCC |
| | 197 420 | GCG GCC |
| | 197 420 | GCG GCC |
| seq.701 | 334 428 499 | GCG GCT GCC |
| | 334 428 499 | GCG GCT GCC |
| | 334 428 499 | GCG GCT GCC |
| seq.702 | 197 414 | GCG GCT |
| | 197 414 | GCG GCT |
| | 197 414 | GCG GCT |
| seq.703 | 30 54 127 | GCG GCC GCT |
| seq.704 | 29 260 | GCG GCG |
| | 29 260 | GCG GCG |
| | 29 260 | GCG GCG |
| seq.706 | 4 484 | GCT GCC |
| | 4 484 | GCT GCC |
| | 4 484 | GCT GCG |
| seq.707 | 258 124 132 | GCC GCC GCC |
| | 258 124 132 | GCC GCC GCC |
| | 258 124 132 | GCC GCC GCC |
| seq.708 | 231 497 | GCC GCC |
| | 231 497 | GCC GCC |
| | 231 497 | GCC GCC |
| seq.709 | 221 258 | GCA GCC |
| | 221 258 | GCA GCC |
| | 221 258 | GCA GCC |
| seq.710 | 234 264 326 | GCG GCG GCC |
| | 234 264 326 | GCG GCG GCC |
| | 234 264 326 | GCG GCG GCC |
| seq.711 | 153 398 | AGC GCG |
| | 153 398 | AGC GCG |
| | 153 398 | AGC GCG |
| seq.712 | 53 216 | GCG GCC |
| seq.713 | 22 382 | GCT GCG |
| | 22 382 | GCT GCG |
| | 22 382 | GCT GCG |
| seq.714 | 231 411 | GCC GCA |
| | 231 411 | GCC GCA |
| | 231 411 | GCC GCA |
| seq.715 | 59 305 | GCG GCC |
| | 59 305 | GCG GCC |
| | 59 305 | GCG GCC |
| seq.716 | 53 231 | GCG GCC |
| | 53 231 | GCG GCC |
| | 53 231 | GCG GCC |
| seq.717 | 258 498 | GCC GCT |
| | 258 498 | GCC GCT |
| | 258 498 | GCC GCT |
| seq.718 | 88 231 | GCC GCC |
| | 88 231 | GCC GCC |
| | 88 231 | GCC GCC |
| seq.719 | 101 363 | GCA GCC |
| | 101 363 | GCA GCC |
| | 101 363 | GCA GCC |
| seq.720 | 354 132 | GCC GCC |
| | 354 132 | GCC GCC |
| | 354 132 | GCC GGC |
| seq.726 | 598 | GAG |
| seq.727 | 598 | AGC |
| seq.728 | 600 | CCG |

The above nucleic acid molecules are provided in plasmids, which are introduced into cells to produce the encoded proteins. The analysis revealed the amino acid positions that affect Rep proteins activities. Changes of amino acids at any of the hit positions result in altered protein activity. Hit positions are numbered and referenced starting from amino acid 1 (nucleotide 321 in AAV-2 genome), also codon 1 of the protein Rep78 coding sequence under control of p5 promoter of AAV-2: 4, 20, 22, 29, 32, 38, 39, 54, 59, 124, 125, 127, 132, 140, 161, 163, 193, 196, 197, 221, 228, 231, 234, 258, 260, 263, 264, 334, 335, 337, 342, 347, 350, 354, 363, 364, 367, 370, 376, 381, 389, 407, 411, 414, 420, 421, 422, 424, 428, 438, 440, 451, 460, 462, 484, 488, 495, 497, 498, 499, 503, 511, 512, 516, 517, 518, 542, 548, 598, 600 and 601. The encoded Rep78, Rep68, Rep 52 and Rep 40 proteins and rAAV encoding the mutant proteins are provided. The corresponding nucleic acid molecules, Rep proteins, rAAV and cells containing the nucleic acid molecules or rAAV in which the native proteins are from other AAV serotypes, including, but are not limited to, AAV-1, AAV-3, AAV-3B, AAV-4, AAV-5 and AAV-6.

Other hit positions identified include: 10, 64, 74, 86, 88, 101, 175, 237, 250, 334, 429 and 519.

Also provided are nucleic acid molecules, the rAAV that encode the mutant proteins, and the encoded proteins in which the native amino acid at each hit position is replaced with another amino acid, or is deleted, or contains additional amino acids at or adjacent to or near the hit positions. In particular the following nucleic acid molecules and rAAV that encode proteins containing the following amino acid replacements or combinations thereof: T by N at Hit position 350; T by I at Hit position 462; P by R at Hit position 497; P by L at Hit position 497; P by Y at Hit position 497; T by N at Hit position 517; L by S at hit position 542; R by S at hit position 548; G by D at Hit position 598; G by S at Hit position 598; V by P at Hit position 600; in order to increase Rep proteins activities in terms on AAV or rAAV productivity. The corresponding nucleic acid molecules, recombinant Rep proteins from the other serotypes and the resulting rAAV are also provided (see FIG. 3 and the above Table for the corresponding position in AAV-1, AAV-3, AAV-3B, AAV-4, AAV-5 and AAV-6).

Mutant adeno-associated virus (AAV) Rep proteins and viruses encoding such proteins that include mutations at one or more of residues 64, 74, 88, 175, 237, 250 and 429, where residue 1 corresponds to residue 1 of the Rep78 protein encoding by nucleotides 321-323 of the AAV-2 genome, and where the amino acids are replaced as follows: L by A at position 64; P by A at position 74; Y by A at position 88; Y by A at position 175; T by A at position 237; T by A at position 250; D by A at position 429 are provided. Nucleic acid molecules encoding these viruses and the mutant proteins are also provided.

Also provided are nucleic acid molecules produced from any of the above-noted nucleic acid molecules by any directed evolution method, including, but are not limited to, re-synthesis, mutagenesis, recombination and gene shuffling and any way by combining any combination of the molecules, i.e., one, two by one, two by two . . . n by n, where n is the number of molecules to be combined (i.e., combining all together). The resulting recombinant AAV and encoded proteins are also provided.

Also provided are nucleic acid molecule in which additional amino acids surrounding each hit, such as one, two, three . . . ten or more, amino acids are systematically replaced, such that the resulting Rep protein(s) has increased or decreased activity. Increased activity as assessed by increased recombinant virus production in suitable cells is of particular interest for production of recombinant viruses for use, for example, in gene therapy.

Also provided are combinations of the above noted mutants in which several of the noted amino acids are changed and optionally additional amino acids surrounding each hit, such as one, two, three . . . ten or more, are replaced.

For all of the mutant proteins provided herein those with increased activity, such as an increase in titer of rAAV when virus containing such mutations and/or expressing such mutant proteins are replicated, are of particular interest. Such mutatations and proteins are provided herein and may be made by the methods herein, including by combining any of the mutations provided herein to produce additional mutant proteins that have altered biological activity, particularly increased activity, compared to the wild-type.

The nucleic acid molecules of SEQ ID Nos. 563-725 and the encoded proteins (SEQ ID Nos. 1-562 and 726-728) are also provided. Recombinant AAV and cells containing the encoding nucleic acids are provided, as are the AAV produced upon replication of the AAV in the cells.

Methods of in vivo or in vitro production of AAV or rAAV using any of the above nucleic acid molecules or cells for intracellular expression of rep proteins or the rep gene mutants are provided. In vitro production is effected using cell free systems, expression or replication and/or virus assembly. In vivo production is effected in mammalian cells that also contain any requisite cis acting elements required for packaging.

Also provided are nucleic acid molecules and rAAV (any serotype) in which position 630 (or the corresponding position in another serotype; see FIG. 3 and the table above). Changes at this position and the region around it lead to changes in the activity or in the quantities of the Rep or Cap proteins and/or the amount of AAV or rAAV produced in cells transduced with AAV encoding such mutants. Such mutations include tgc to gcg change (SEQ ID No. 721). Mutations at any position surrounding the codon position 630 that increase or decrease the Rep or Cap proteins quantities or activities are also provided. Methods using the rAAV (any serotype) that contain nucleic acid molecules with a mutation at position 630 or within 1, 2, 3 . . . 10 or more bases thereof for the intracellular expression rep proteins or the rep gene mutants covered by claims 10 to 13, for the production of AAV or rAAV (either in vitro, in vivo or ex vivo) are provided. In vitro methods include cell free systems, expression or replication and/or virus assembly.

Also provided are rAAV (and other serotypes with corresponding changes) and nucleic acid molecules encoding an amino acid replacement by N at Hit position 350 of AAV-1, AAV-3, AAV-3B, AAV-4 and AAV-6 or at Hit position 346 of AAV-5; by I at Hit position 462 of AAV-1, AAV-3, AAV-3B, AAV-4 and AAV-6 or at Hit position 458 of AAV-5; by either R, L or Y at Hit position 497 of AAV-1, AAV-3, AAV-3B, AAV-4 and AAV-6 or at Hit position 493 of AAV-5; by N at Hit position 517 of AAV-1, AAV-3, AAV-3B, AAV-4 and AAV-6 or at Hit position 535 of AAV-5; by S at hit position 543 of AAV-1 and AAV-6 or at hit position 542 of AAV-3, AAV-3B and AAV-4 or at hit position 561 of AAV-5; by S at hit position 549 of AAV-1 and AAV-6 or at hit position 548 of AAV-3, AAV-3B and AAV-4 or at hit position 567 of AAV-5; by either D or S at Hit position 599 of AAV-1, AAV-4 and AAV-6 or at Hit position 600 of AAV-3 and AAV-3B; by P at Hit position 602 of AAV-1, AAV-4 and AAV-6 or at hit position 603 of AAV-3 and AAV-3B or at hit position 589 of AAV-5 in order to increase Rep proteins activities as assessed by AAV or rAAV productivity Methods using such AAV for expression of the encoded proteins and production of AAV are also provided.

Since modifications will be apparent to those of skill in this art, it is intended that this invention be limited only by the scope of the appended claims.

What is claimed is:

1. An adeno-associated virus (AAV), comprising nucleic acid encoding the sequence of amino acids in any of SEQ ID Nos. 1-562 and 726-728 or encoding a sequence of amino acids encoded by SEQ ID Nos. 722-725.

2. The AAV of claim 1, wherein the sequence of nucleotides encoding the sequence of amino acids is set forth in SEQ ID Nos. 563-725.

3. The AAV of claim 1 that has an altered activity in a Rep protein and/or a capsid protein.

4. The AAV of claim 3, wherein the alteration leads to greater activity in the Rep gene manifested as an increased titer of virus upon introduction and replication in a host cell compared to the titer of virus upon introduction and replication of a wild type Rep gene.

5. The AAV of claim 1 that is of serotype AAV-1, AAV-2, AAV-3, AAV-3B, AAV-4, AAV-5 or AAV-6.

6. A mutant adeno-associate virus (AAV) Rep protein, comprising mutations at one or more of residues 4, 20, 22, 29, 32, 38, 39, 54, 59, 124, 125, 127, 132, 140, 161, 163, 193, 196, 197, 221, 228, 231, 234, 258, 260, 263, 264, 334, 335, 337, 342, 347, 350, 354, 363, 364, 367, 370, 376, 381, 389, 407, 411, 414, 420, 421, 422, 424, 428, 438, 440, 451, 460, 462, 484, 488, 495, 497, 498, 499, 503, 511, 512, 516, 517, 518, 542, 548, 598, 600 and 601 of AAV-2 or the corresponding residues in other serotypes, wherein residue 1 corresponds to residue 1 of the Rep78 protein encoding by nucleotides 321-323 of the AAV-2 genome, wherein the mutations comprise insertions, deletions or replacements of the native amino acid residue(s).

7. The Rep protein of claim 6 that is Rep 78, Rep 68, Rep 52 or Rep 40.

8. The mutant AAV Rep protein of claim 6, wherein the AAV is an AAV-1, AAV-2, AAV-3, AAV-3b, AAV-4, AAV-5 or AAV-6, wherein the mutation is in the equivalent position in each serotype, wherein the listed residues are the positions in AAV-2.

9. A mutant AAV Rep protein of claim 6 that has increased activity compared to the native protein, wherein activity is assessed by measuring viral production when an AAV that encodes the protein is introduced into a cell under conditions wherein the virus replications.

10. A mutant AAV Rep protein of claim 6 that has decreased activity compared to the native protein, wherein activity is assessed by measuring viral production when an AAV that encodes the protein is introduced into a cell under conditions wherein the virus replicates.

11. A mutant Rep protein of claim 6, further comprising a mutation at one or more of residues 10, 64, 74, 86, 88, 101, 175, 237, 250, 334, 429 and 519.

12. The mutant Rep protein of claim 6, wherein the amino acids are replaced as follows: T by N at position 350; T by I at position 462; P by R at position 497; P by L at position 497; P by Y at position 497; T by N at position 517; G by D at position 598; G by S at position 598; V by P at position 600, whereby the activity of the Rep protein is increased as assessed by rAAV production compared to the native Rep protein.

13. A mutant Rep protein of claim 6, comprising two or more of the mutations.

14. A mutant adeno-associate virus (AAV) Rep protein, comprising mutations at one or more of residues 64, 74, 88, 175, 237, 250 and 429, wherein: residue 1 corresponds to residue 1 of the Rep78 protein encoding by nucleotides 321-323 of the AAV-2 genome;
wherein the amino acids are replaced as follows:
L by A at position 64;
P by A at position 74;
Y by A at position 88;
Y by A at position 175;
T by A at position 237;
T by A at position 250;
D by A at position 429;
the mutations comprise insertions, deletions or replacements of the native amino acid residue.

15. A nucleic acid molecule encoding the protein of claim 6.

16. A recombinant AAV comprising the nucleic acid molecule of claim 15.

17. A eukaryotic cell, comprising the recombinant AAV of claim 16.

18. A collection of nucleic acid molecules comprising a plurality of the molecules of claim 17.

19. A collection of nucleic acid molecules comprising a plurality of the molecules of claim 15.

20. An isolated nucleic acid molecule encoding the proteins of SEQ ID Nos. 1-562 and 726-728 or encoding a sequence of amino acids encoded by SEQ ID Nos. 722-725.

21. A Rep protein of any of SEQ ID Nos. 1-562 and 726-728 or encoding a sequence of amino acids encoded by SEQ ID Nos. 722-725.

22. A Rep protein encoded by any of SEQ ID Nos. 564-725.

23. A method for intracellular expression of a mutant Rep protein, comprising:
introducing the recombinant AAV of claim 16 into a host cell; and
culturing the cell, under conditions and in which the AAV Rep proteins are expressed.

24. The method of claim 23, wherein the AAV replicate.

25. An AAV genome, comprising a mutation at one or more of nucleotides corresponding to nucleotides 2209-2211 of the AAV-2 genome, which encode amino acid residue 630 of the Rep78 protein, wherein:
the mutation is a deletion, insertion or replacement of a nucleotide;
and the mutation results in a change in the activity or in the quantities of the Rep or Cap proteins as assessed by the level of replication of the AAV genome.

26. The AAV genome of claim 25, wherein the mutation at position 630 is a tgc to gcg and the intron comprises the sequence (SEQ ID No. 722):
gtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggcgcgcgatggttatcttccag.

27. The AAV genome of claim 25, wherein the mutation at position 630 is a tgc to cgc and the intron comprises the sequence (SEQ ID No. 723):
gtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgg ccgccgatggttatcttccag.

28. The AAV genome of claim 25, wherein the mutation at position 630 is a tgc to cct and the intron comprises the sequence (SEQ ID No. 724):
gtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggccctcgatggttatcttccag.

29. The AAV genome of claim 25, wherein the mutation at position 630 is a tgc to tca and the intron comprises the sequence (SEQ ID No.725):
gtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctcacgatggttatcttccag.

30. A method for intracellular expression of a mutant Rep protein, comprising:
introducing the recombinant AAV of claim 25 into a host cell; and
culturing the cell, under conditions and in which the AAV Rep proteins and/or cap proteins are expressed.

31. The method of claim 30, wherein the AAV replicate.

32. The AAV genome of claim 25, wherein the AAV is of serotype AAV-1, AAV-3, AAV-3B, AAV-4, AAV-5 or AAV-6.

33. A method of titering virus by a method designated tagged replication and expression enhancement, comprising:
(i) incubating host cells with a reporter virus vector and with a titering virus of unknown titer, wherein a titering virus increases or decreases the output signal from the reporter virus; and
(ii) measuring the output signal of the reporter virus in and determining the titer of the reporter virus; and
(ii) determining the titer of the titering virus by comparing the titer of the reporter virus in the presence and absence of the titering virus.

34. A process for the production of an adeno-associated virus (AAV) protein or a recombinant AAV having a predetermined property, comprising:
(a) producing a population of sets of nucleic acid molecules that encode modified forms of a target protein;
(b) introducing each set of nucleic acid molecules into host cells and expressing the encoded protein, wherein the host cells are present in an addressable array;
(c) individually screening the sets of encoded proteins to identify one or more proteins that have activity that differs from the target protein, wherein each such protein is designated a hit;
(d) modifying the nucleic acid molecules that encode the hits, to produce a set of nucleic acid molecules that encode modified hits, wherein the nucleic acid molecules comprise rAAV vectors;
(e) introducing the each set nucleic acids that encode the modified hits into cells; and
(f) individually screening the sets cells that contain the nucleic acid molecules that encode the modified hits to identify one or more cells that encodes a protein that has activity that differs from the target protein and has properties that differ from the original hits, wherein each such protein is designated a lead.

35. The process of claim 34, wherein the cells are eukaryotic cells that are transduced with the vectors.

36. The method of claim 35, wherein at step (f) the titer of the viral vectors in each set of cells is determined.

37. The method of claim 36, wherein the target protein is a protein involved in viral replication.

38. The method of claim 37, wherein the target protein is a Rep protein.

39. The AAV mutant Rep protein of claim 6 binds to a sequence from a papillomavirus, oncogene or human immunodeficiency virus (HIV) with different affinity from a wild-type AAV Rep protein.

40. A fusion protein, comprising the tat protein of HIV and the mutant Rep protein of claim 39.

41. The fusion protein of claim 40, wherein the HIV is HIV-1.

42. A pharmaceutical composition, comprising the protein of claim 39 in a pharmaceutically acceptable carrier.

43. A recombinant adeno-associated virus (rAAV) that encodes a mutant Rep protein that has increased activity, wherein increased activity of a Rep protein is manifested as an increased titer of virus upon introduction and replication in a host cell compared to the titer of virus upon introduction and replication of a wild type Rep gene.

44. A mutant AAV Rep protein that has increased activity, wherein increased activity of a Rep protein is manifested as an increased titer of virus upon introduction and replication in a host cell compared to the titer of virus upon introduction and replication of a wild type Rep gene.

45. A nucleic acid molecule that encodes that mutant Rep protein of claim 44.

46. A cell, comprising the nucleic acid molecule of claim 45.

47. A rAAV, comprising the nucleic acid molecule of claim 45.

48. A cell, comprising the rAAV of claim 47.

49. A method for production of rAAV, comprising:
introducing the rAAV of claim 47 into a cell under conditions whereby the virus replicates to produce encapsulated rAAV.

50. A method for the production of mutant Rep protein comprising expressing the nucleic acid molecule of claim 45.

51. The method of claim 50, wherein expression is effected in vivo.

52. The method of claim 50, wherein expression is effected in vitro.

53. A method for producing Rep protein in a host cell, comprising:
expressing the protein encoded by the nucleic acid encoding the protein of claim 44, wherein the method is performed in vitro or in vivo.

54. The method of claim 53, wherein the nucleic acid is introduced into a cell.

55. The method of claim 53, wherein expression is effected in a cell-free system.

56. A method of treating or inhibiting infection by human papilloma virus or a human immunodeficiency virus, comprising administering, to a subject exposed to the virus or infected with the virus, a composition containing a rAAV of claim 47.

57. A nucleic acid molecule encoding the protein of claim 7.

58. A nucleic acid molecule encoding the protein of claim 8.

59. A nucleic acid molecule encoding the protein of claim 9.

60. A nucleic acid molecule encoding the protein of claim 10.

61. A nucleic acid molecule encoding the protein of claim 11.

62. A nucleic acid molecule encoding the protein of claim 12.

63. A nucleic acid molecule encoding the protein of claim 13.

64. A nucleic acid molecule encoding the protein of claim 14.

65. A recombinant AAV comprising the nucleic acid molecule of claim 57.

66. A recombinant AAV comprising the nucleic acid molecule of claim 58.

67. A recombinant AAV comprising the nucleic acid molecule of claim 59.

68. A recombinant AAV comprising the nucleic acid molecule of claim 60.

69. A recombinant AAV comprising the nucleic acid molecule of claim 61.

70. A recombinant AAV comprising the nucleic acid molecule of claim 62.

71. A recombinant AAV comprising the nucleic acid molecule of claim 63.

72. A recombinant AAV comprising the nucleic acid molecule of claim 64.

73. A cell, comprising the recombinant AAV of claim 65.

74. A cell, comprising the recombinant AAV of claim 66.

75. A cell, comprising the recombinant AAV of claim 67.

76. A cell, comprising the recombinant AAV of claim 68.

77. A cell, comprising the recombinant AAV of claim 69.

78. A cell, comprising the recombinant AAV of claim 70.

79. A cell, comprising the recombinant AAV of claim 71.

80. A cell, comprising the recombinant AAV of claim 72.

81. A method for intracellular expression of a mutant Rep protein, comprising:
culturing the cell of claim 73 under conditions and in which the AAV Rep proteins are expressed.

82. The method of claim 81, wherein the AAV replicate.

83. A method for intracellular expression of a mutant Rep protein, comprising culturing the cell of claim 74 under conditions in which the AAV Rep proteins are expressed.

84. The method of claim 83, wherein the AAV replicate.

85. A method of altering expression of a gene, comprising contacting the gene with a mutant rep protein that has increased activity, wherein increased activity of a Rep protein is manifested as an increased titer of virus upon introduction and replication in a host cell compared to the titer of virus upon introduction and replication of a wild type Rep gene.

86. The method of claim 85, wherein the gene is a viral gene.

87. The method of claim 85, wherein the gene is a cellular gene.

88. The mutant protein of claim 6, wherein serotype is AAV-1, AAV-2, AAV-3, AAV-3B, AAV-4, AAV-5 or AAV-6.

89. The protein of claim 44, wherein the mutation is at a residue corresponding to one or more of residues 350, 462, 497, 517, 542, 548, 598, 600 and 630 of AAV-2.

90. The mutant protein of claim 89, wherein serotype is AAV-1, AAV-2, AAV-3, AAV-3B, AAV-4, AAV-5 or AAV-6.

91. The AAV mutant Rep protein of claim 44 that binds to a sequence from a papillomavirus, oncogene or human immunodeficiency virus (HIV) with different affinity from a wild-type AAV Rep protein.

92. A pharmaceutical composition, comprising the protein of claim 91 in a pharmaceutically acceptable carrier.

93. A pharmaceutical composition, comprising the rAAV of claim 47 in a pharmaceutically acceptable carrier.
